SEQUENCE LISTING (258 sequences)

SEQ ID NO: 1 - DNA, 26 bp, Artificial Sequence
  Primer
cacgtctcccaaatgcttgagtgacg

SEQ ID NO: 2 - DNA, 20 bp, Artificial Sequence
  Primer
cctcgaggccagggcgttcc

SEQ ID NO: 3 - DNA, 42 bp, Artificial Sequence
  Primer
tcacttgcttccgttgaggtcggggaccaagacctaatcaga

SEQ ID NO: 4 - DNA, 43 bp, Artificial Sequence
  Primer
ggtttcggatgttacagcgtagccgcaggaagaagagtcacag

SEQ ID NO: 5 - DNA, 38 bp, Artificial Sequence
  Primer
tcacttgcttccgttgaggaggccagggcgttccaatc

SEQ ID NO: 6 - DNA, 41 bp, Artificial Sequence
  Primer
ggtttcggatgttacagcgtcaatagcgcgagggcagtttc

SEQ ID NO: 7 - DNA, 42 bp, Artificial Sequence
  Primer
tcacttgcttccgttgaggggcacccgcaatcctaataacaa

SEQ ID NO: 8 - DNA, 43 bp, Artificial Sequence; identical to SEQ ID NO: 4
  Primer

SEQ ID NO: 9 - DNA, 23 bp, Artificial Sequence
  Primer
tcggggaccaagacctaatcaga

SEQ ID NO: 10 - DNA, 23 bp, Artificial Sequence
  Primer
agccgcaggaagaagagtcacag

SEQ ID NO: 11 - DNA, 19 bp, Artificial Sequence
  Primer
aggccagggcgttccaatc

SEQ ID NO: 12 - DNA, 21 bp, Artificial Sequence
  Primer
caatagcgcgagggcagtttc

SEQ ID NO: 13 - DNA, 23 bp, Artificial Sequence
  Primer
ggcacccgcaatcctaataacaa

SEQ ID NO: 14 - DNA, 23 bp, Artificial Sequence
  Primer
agccgcaggaagaagagtcacag

SEQ ID NO: 15 - DNA, 28 bp, Artificial Sequence
  Primer
acatcacagcttctacacccgttaaggt

SEQ ID NO: 16 - DNA, 28 bp, Artificial Sequence
  Primer
atacagaatacatagattgctgttatcc

SEQ ID NO: 17 - DNA, 27 bp, Artificial Sequence
  Primer
gcatcgttgactatggtgtccgattct

SEQ ID NO: 18 - DNA, 28 bp, Artificial Sequence
  Primer
gctgcattggtttgttatatcgttatgc

SEQ ID NO: 19 - DNA, 42 bp, Artificial Sequence
  Primer
tcacttgcttccgttgaggagccgcttgtcacaatgccaatt

SEQ ID NO: 20 - DNA, 43 bp, Artificial Sequence
  Primer
ggtttcggatgttacagcgtcatcaccaagctcgccaacagtt

SEQ ID NO: 21 - DNA, 43 bp, Artificial Sequence
  Primer
tcacttgcttccgttgaggaggttgccatcattttggcatctt

SEQ ID NO: 22 - DNA, 43 bp, Artificial Sequence
  Primer
ggtttcggatgttacagcgtctttgcgccagcgatagtgactt

SEQ ID NO: 23 - DNA, 41 bp, Artificial Sequence
  Primer
tcacttgcttccgttgaggatggcacccgtttctgcaatgg

SEQ ID NO: 24 - DNA, 44 bp, Artificial Sequence
  Primer
ggtttcggatgttacagcgttcgggcagctgacacgaatgtaga

SEQ ID NO: 25 - DNA, 43 bp, Artificial Sequence
  Primer
tcacttgcttccgttgagggaatggcgatgtagtggctattga

SEQ ID NO: 26 - DNA, 43 bp, Artificial Sequence
  Primer
ggtttcggatgttacagcgttaatgccggcatccaaacataat

SEQ ID NO: 27 - DNA, 43 bp, Artificial Sequence
  Primer
tcacttgcttccgttgaggtagccagcgtggtggttcatacaa

SEQ ID NO: 28 - DNA, 43 bp, Artificial Sequence
  Primer
ggtttcggatgttacagcgtctcccggcagaaagctgtaagct

SEQ ID NO: 29 - DNA, 42 bp, Artificial Sequence
  Primer
tcacttgcttccgttgaggtatagagcccgtgctggtgatgc

SEQ ID NO: 30 - DNA, 44 bp, Artificial Sequence
  Primer
ggtttcggatgttacagcgtatcgccattcaagtctgggaagaa

SEQ ID NO: 31 - DNA, 43 bp, Artificial Sequence
  Primer
tcacttgcttccgttgaggtggctcaggccatactggcattac

SEQ ID NO: 32 - DNA, 43 bp, Artificial Sequence
  Primer
ggtttcggatgttacagcgttttgcgccagcgatagtgacttg

SEQ ID NO: 33 - DNA, 42 bp, Artificial Sequence
  Primer
tcacttgcttccgttgaggttcccgtcaggcaaagttgaagg

SEQ ID NO: 34 - DNA, 44 bp, Artificial Sequence
  Primer
ggtttcggatgttacagcgtgacggcaattcctgtttgagcaga

SEQ ID NO: 35 - DNA, 23 bp, Artificial Sequence
  Primer
agccgcttgtcacaatgccaatt

SEQ ID NO: 36 - DNA, 23 bp, Artificial Sequence
  Primer
catcaccaagctcgccaacagtt

SEQ ID NO: 37 - DNA, 24 bp, Artificial Sequence
  Primer
aggttgccatcattttggcatctt

SEQ ID NO: 38 - DNA, 23 bp, Artificial Sequence
  Primer
ctttgcgccagcgatagtgactt

SEQ ID NO: 39 - DNA, 22 bp, Artificial Sequence
  Primer
atggcacccgtttctgcaatgg

SEQ ID NO: 40 - DNA, 24 bp, Artificial Sequence
  Primer
tcgggcagctgacacgaatgtaga

SEQ ID NO: 41 - DNA, 24 bp, Artificial Sequence
  Primer
gaatggcgatgtagtggctattga

SEQ ID NO: 42 - DNA, 23 bp, Artificial Sequence
  Primer
taatgccggcatccaaacataat

SEQ ID NO: 43 - DNA, 24 bp, Artificial Sequence
  Primer
tagccagcgtggtggttcatacaa

SEQ ID NO: 44 - DNA, 23 bp, Artificial Sequence
  Primer
ctcccggcagaaagctgtaagct

SEQ ID NO: 45 - DNA, 23 bp, Artificial Sequence
  Primer
tatagagcccgtgctggtgatgc

SEQ ID NO: 46 - DNA, 24 bp, Artificial Sequence
  Primer
atcgccattcaagtctgggaagaa

SEQ ID NO: 47 - DNA, 24 bp, Artificial Sequence
  Primer
tggctcaggccatactggcattac

SEQ ID NO: 48 - DNA, 23 bp, Artificial Sequence
  Primer
tttgcgccagcgatagtgacttg

SEQ ID NO: 49 - DNA, 23 bp, Artificial Sequence
  Primer
ttcccgtcaggcaaagttgaagg

SEQ ID NO: 50 - DNA, 24 bp, Artificial Sequence
  Primer
gacggcaattcctgtttgagcaga

SEQ ID NO: 51 - DNA, 26 bp, Artificial Sequence
  Primer
ccagctccaataggaatgtcgcactc

SEQ ID NO: 52 - DNA, 28 bp, Artificial Sequence
  Primer
tccgcagatgtacatattacaatctacg

SEQ ID NO: 53 - DNA, 23 bp, Artificial Sequence
  Primer
ttaaatgcaccggccacggtttg

SEQ ID NO: 54 - DNA, 24 bp, Artificial Sequence
  Primer
atagcgccaggacaaactggtgtt

SEQ ID NO: 55 - DNA, 24 bp, Artificial Sequence
  Primer
tatatgcgccaagctggtgtgagt

SEQ ID NO: 56 - DNA, 24 bp, Artificial Sequence
  Primer
cgaggcggaggtacaaattgacag

SEQ ID NO: 57 - DNA, 24 bp, Artificial Sequence
  Primer
atgaagccgagccaaacataccaa

SEQ ID NO: 58 - DNA, 40 bp, Artificial Sequence
  Primer
tcacttgcttccgttgaggatgcaccggccacggtttgtg

SEQ ID NO: 59 - DNA, 44 bp, Artificial Sequence
  Primer
ggtttcggatgttacagcgtatgcgccaagctggtgtgagttga

SEQ ID NO: 60 - DNA, 43 bp, Artificial Sequence
  Primer
tcacttgcttccgttgaggtgctggcgctgctcttcaaatacc

SEQ ID NO: 61 - DNA, 40 bp, Artificial Sequence
  Primer
ggtttcggatgttacagcgtcggggctgcttgtgggaagg

SEQ ID NO: 62 - DNA, 43 bp, Artificial Sequence
  Primer
tcacttgcttccgttgaggatagcgccaggacaaactggtgtt

SEQ ID NO: 63 - DNA, 44 bp, Artificial Sequence
  Primer
ggtttcggatgttacagcgttatatgcgccaagctggtgtgagt

SEQ ID NO: 64 - DNA, 43 bp, Artificial Sequence
  Primer
tcacttgcttccgttgaggcgaggcggaggtacaaattgacag

SEQ ID NO: 65 - DNA, 44 bp, Artificial Sequence
  Primer
ggtttcggatgttacagcgtatgaagccgagccaaacataccaa

SEQ ID NO: 66 - DNA, 21 bp, Artificial Sequence
  Primer
atgcaccggccacggtttgtg

SEQ ID NO: 67 - DNA, 24 bp, Artificial Sequence
  Primer
atgcgccaagctggtgtgagttga

SEQ ID NO: 68 - DNA, 24 bp, Artificial Sequence
  Primer
tgctggcgctgctcttcaaatacc

SEQ ID NO: 69 - DNA, 20 bp, Artificial Sequence
  Primer
cggggctgcttgtgggaagg

SEQ ID NO: 70 - DNA, 56 bp, Artificial Sequence
  Primer
ttaccctaatatgtttatcacccgcgaagaagctattcgtcacgttcgtgcgtgga

SEQ ID NO: 71 - DNA, 56 bp, Artificial Sequence
  Primer
ctgacaagtatgtccgcaatctacaacacaggctctatgagtgtctctatagaaat

SEQ ID NO: 72 - DNA, 56 bp, Artificial Sequence
  Primer
cataacacttgctgtaacttatcacaccgtttctacaggttagctaacgagtgtgc

SEQ ID NO: 73 - DNA, 50 bp, Artificial Sequence
  Primer
ttaccctaatatgtttatcacccgcgaagaagctattcgtcacgttcgtg

SEQ ID NO: 74 - DNA, 70 bp, Artificial Sequence
  Primer
gcgttctcttaaagctcctgccgtagtgtcagtatcatcaccagatgctgttactacatataatggatac

SEQ ID NO: 75 - DNA, 70 bp, Artificial Sequence
  Primer
ctttggctggctcttacagagattggtcctattcaggacagcgtacagagttaggtgttgaatttcttaa

SEQ ID NO: 76 - DNA, 70 bp, Artificial Sequence
  Primer
ctacgtagtgaagctttcgagtactaccatactcttgatgagagttttcttggtaggtacatgtctgctt

SEQ ID NO: 77 - DNA, 70 bp, Artificial Sequence
  Primer
tgccaattggttatgtgacacatggttttaatcttgaagaggctgcgcgctgtatgcgttctcttaaagc

SEQ ID NO: 78 - DNA, 70 bp, Artificial Sequence
  Primer
tataaagttaccaagggaaagcccgtaaaaggtgcttggaacattggacaacagagatcagttttaacac

SEQ ID NO: 79 - DNA, 69 bp, Artificial Sequence
  Primer
tgcttcattgatgttgttaacaaggcactcgaaatgtgcattgatcaagtcactatcgctggcgcaaag

SEQ ID NO: 80 - DNA, 70 bp, Artificial Sequence
  Primer
tgtcgacgccatggtttatacttcagacctgctcaccaacagtgtcattattatggcatatgtaactggt

SEQ ID NO: 81 - DNA, 70 bp, Artificial Sequence
  Primer
tactgttgaaaaactcaggcctatctttgaatggattgaggcgaaacttagtgcaggagttgaatttctc

SEQ ID NO: 82 - DNA, 61 bp, Artificial Sequence
  Primer
acctattctgttgcttgaccaagctcttgtatcagacgttggagatagtactgaagtttcc

SEQ ID NO: 83 - DNA, 70 bp, Artificial Sequence
  Primer
gcctattaatgtcatagtttttgatggcaagtccaaatgcgacgagtctgcttctaagtctgcttctgtg

SEQ ID NO: 84 - DNA, 70 bp, Artificial Sequence
  Primer
tgagagctaacaacactaaaggttcactgcctattaatgtcatagtttttgatggcaagtccaaatgcga

SEQ ID NO: 85 - DNA, 70 bp, Artificial Sequence
  Primer
acttgcatgatgtgctataagcgcaatcgtgccacacgcgttgagtgtacaactattgttaatggcatga

SEQ ID NO: 86 - DNA, 70 bp, Artificial Sequence
  Primer
ggcgatgtagtggctattgactatagacactattcagcgagtttcaagaaaggtgctaaattactgcata

SEQ ID NO: 87 - DNA, 70 bp, Artificial Sequence
  Primer
tcaaaccaaacacttggtgtttacgttgtctttggagtacaaagccagtagatacttcaaattcatttga

SEQ ID NO: 88 - DNA, 63 bp, Artificial Sequence
  Primer
tagtgctgttggcaacatttgctacacaccttccaaactcattgagtatagtgattttgctac

SEQ ID NO: 89 - DNA, 70 bp, Artificial Sequence
  Primer
tcatagctaacatctttactcctcttgtgcaacctgtgggtgctttagatgtgtctgcttcagtagtggc

SEQ ID NO: 90 - DNA, 70 bp, Artificial Sequence
  Primer
ggtattattgccatattggtgacttgtgctgcctactactttatgaaattcagacgtgtttttggtgagt

SEQ ID NO: 91 - DNA, 70 bp, Artificial Sequence
  Primer
gtgatgtcagagaaactatgacccatcttctacagcatgctaatttggaatctgcaaagcgagttcttaa

SEQ ID NO: 92 - DNA, 70 bp, Artificial Sequence
  Primer
aaccatcaagcctgtgtcgtataaactcgatggagttacttacacagagattgaaccaaaattggatggg

SEQ ID NO: 93 - DNA, 70 bp, Artificial Sequence
  Primer
gttttctacaaggaaacatcttacactacaaccatcaagcctgtgtcgtataaactcgatggagttactt

SEQ ID NO: 94 - DNA, 70 bp, Artificial Sequence
  Primer
ccttgaatgaggatctccttgagatactgagtcgtgaacgtgttaacattaacattgttggcgattttca

SEQ ID NO: 95 - DNA, 70 bp, Artificial Sequence
  Primer
gccatggtttatacttcagacctgctcaccaacagtgtcattattatggcatatgtaactggtggtcttg

SEQ ID NO: 96 - DNA, 70 bp, Artificial Sequence
  Primer
caacagacttctcagtggttgtctaatcttttgggcactactgttgaaaaactcaggcctatctttgaat

SEQ ID NO: 97 - DNA, 56 bp, Artificial Sequence
  Primer
ttcccgtcaggcaaagttgaagggtgcatggtacaagtaacctgtggaactacaac

SEQ ID NO: 98 - DNA, 56 bp, Artificial Sequence
  Primer
ggttcaccatctggtgtttatcagtgtgccatgagacctaatcataccattaaagg

SEQ ID NO: 99 - DNA, 52 bp, Artificial Sequence
  Primer
agatcatgttgacatattgggacctctttctgctcaaacaggaattgccgtc

SEQ ID NO: 100 - DNA, 70 bp, Artificial Sequence
  Primer
taaaaaggacaaaaagaaaaagactgatgaagctcagcctttgccgcagagacaaaagaagcagcccact

SEQ ID NO: 101 - DNA, 70 bp, Artificial Sequence
  Primer
acggcaaaatgaaagagctcagccccagatggtacttctattacctaggaactggcccagaagcttcact

SEQ ID NO: 102 - DNA, 70 bp, Artificial Sequence
  Primer
ggcgctaacaaagaaggcatcgtatgggttgcaactgagggagccttgaatacacccaaagaccacattg

SEQ ID NO: 103 - DNA, 70 bp, Artificial Sequence
  Primer
gtccagatgaccaaattggctactaccgaagagctacccgacgagttcgtggtggtgacggcaaaatgaa

SEQ ID NO: 104 - DNA, 70 bp, Artificial Sequence
  Primer
gaggtggtgaaactgccctcgcgctattgctgctagacagattgaaccagcttgagagcaaagtttctgg

SEQ ID NO: 105 - DNA, 70 bp, Artificial Sequence
  Primer
aaaagaaaaagactgatgaagctcagcctttgccgcagagacaaaagaagcagcccactgtgactcttct

SEQ ID NO: 106 - DNA, 70 bp, Artificial Sequence
  Primer
aaattgcacaatttgctccaagtgcctctgcattctttggaatgtcacgcattggcatggaagtcacacc

SEQ ID NO: 107 - DNA, 70 bp, Artificial Sequence
  Primer
accaatttaacaaggcgattagtcaaattcaagaatcacttacaacaacatcaactgcattgggcaagct

SEQ ID NO: 108 - DNA, 70 bp, Artificial Sequence
  Primer
cacctggaacaaatgcttcatctgaagttgctgttctatatcaagatgttaactgcactgatgtttctac

SEQ ID NO: 109 - DNA, 70 bp, Artificial Sequence
  Primer
aaagggctaccaccttatgtccttcccacaagcagccccgcatggtgttgtcttcctacatgtcacgtat

SEQ ID NO: 110 - DNA, 70 bp, Artificial Sequence
  Primer
tcaggaaattgtgatgtcgttattggcatcattaacaacacagtttatgatcctctgcaacctgagcttg

SEQ ID NO: 111 - DNA, 70 bp, Artificial Sequence
  Primer
ttgatcttggcgacatttcaggcattaacgcttctgtcgtcaacattcaaaaagaaattgaccgcctcaa

SEQ ID NO: 112 - DNA, 70 bp, Artificial Sequence
  Primer
gaggaacttcaccacagcgccagcaatttgtcatgaaggcaaagcatacttccctcgtgaaggtgttttt

SEQ ID NO: 113 - DNA, 56 bp, Artificial Sequence; identical to SEQ ID NO: 70
  Primer

SEQ ID NO: 114 - DNA, 56 bp, Artificial Sequence; identical to SEQ ID NO: 71
  Primer

SEQ ID NO: 115 - DNA, 56 bp, Artificial Sequence; identical to SEQ ID NO: 72
  Primer

SEQ ID NO: 116 - DNA, 50 bp, Artificial Sequence; identical to SEQ ID NO: 73
  Primer

SEQ ID NO: 117 - DNA, 70 bp, Artificial Sequence; identical to SEQ ID NO: 74
  Primer

SEQ ID NO: 118 - DNA, 70 bp, Artificial Sequence; identical to SEQ ID NO: 75
  Primer

SEQ ID NO: 119 - DNA, 70 bp, Artificial Sequence; identical to SEQ ID NO: 76
  Primer

SEQ ID NO: 120 - DNA, 70 bp, Artificial Sequence; identical to SEQ ID NO: 77
  Primer

SEQ ID NO: 121 - DNA, 70 bp, Artificial Sequence; identical to SEQ ID NO: 78
  Primer

SEQ ID NO: 122 - DNA, 69 bp, Artificial Sequence; identical to SEQ ID NO: 79
  Primer

SEQ ID NO: 123 - DNA, 70 bp, Artificial Sequence; identical to SEQ ID NO: 80
  Primer

SEQ ID NO: 124 - DNA, 70 bp, Artificial Sequence; identical to SEQ ID NO: 81
  Primer

SEQ ID NO: 125 - DNA, 61 bp, Artificial Sequence; identical to SEQ ID NO: 82
  Primer

SEQ ID NO: 126 - DNA, 70 bp, Artificial Sequence; identical to SEQ ID NO: 83
  Primer

SEQ ID NO: 127 - DNA, 70 bp, Artificial Sequence; identical to SEQ ID NO: 84
  Primer

SEQ ID NO: 128 - DNA, 70 bp, Artificial Sequence; identical to SEQ ID NO: 85
  Primer

SEQ ID NO: 129 - DNA, 70 bp, Artificial Sequence; identical to SEQ ID NO: 86
  Primer

SEQ ID NO: 130 - DNA, 70 bp, Artificial Sequence; identical to SEQ ID NO: 87
  Primer

SEQ ID NO: 131 - DNA, 63 bp, Artificial Sequence; identical to SEQ ID NO: 88
  Primer

SEQ ID NO: 132 - DNA, 70 bp, Artificial Sequence; identical to SEQ ID NO: 89
  Primer

SEQ ID NO: 133 - DNA, 70 bp, Artificial Sequence; identical to SEQ ID NO: 90
  Primer

SEQ ID NO: 134 - DNA, 70 bp, Artificial Sequence; identical to SEQ ID NO: 91
  Primer

SEQ ID NO: 135 - DNA, 70 bp, Artificial Sequence; identical to SEQ ID NO: 92
  Primer

SEQ ID NO: 136 - DNA, 70 bp, Artificial Sequence; identical to SEQ ID NO: 93
  Primer

SEQ ID NO: 137 - DNA, 70 bp, Artificial Sequence; identical to SEQ ID NO: 94
  Primer

SEQ ID NO: 138 - DNA, 70 bp, Artificial Sequence; identical to SEQ ID NO: 95
  Primer

SEQ ID NO: 139 - DNA, 70 bp, Artificial Sequence; identical to SEQ ID NO: 96
  Primer

SEQ ID NO: 140 - DNA, 56 bp, Artificial Sequence; identical to SEQ ID NO: 97
  Primer

SEQ ID NO: 141 - DNA, 56 bp, Artificial Sequence; identical to SEQ ID NO: 98
  Primer

SEQ ID NO: 142 - DNA, 52 bp, Artificial Sequence; identical to SEQ ID NO: 99
  Primer

SEQ ID NO: 143 - DNA, 70 bp, Artificial Sequence; identical to SEQ ID NO: 100
  Primer

SEQ ID NO: 144 - DNA, 70 bp, Artificial Sequence; identical to SEQ ID NO: 101
  Primer

SEQ ID NO: 145 - DNA, 70 bp, Artificial Sequence; identical to SEQ ID NO: 102
  Primer

SEQ ID NO: 146 - DNA, 70 bp, Artificial Sequence; identical to SEQ ID NO: 103
  Primer

SEQ ID NO: 147 - DNA, 70 bp, Artificial Sequence; identical to SEQ ID NO: 104
  Primer

SEQ ID NO: 148 - DNA, 70 bp, Artificial Sequence; identical to SEQ ID NO: 105
  Primer

SEQ ID NO: 149 - DNA, 70 bp, Artificial Sequence; identical to SEQ ID NO: 106
  Primer

SEQ ID NO: 150 - DNA, 70 bp, Artificial Sequence; identical to SEQ ID NO: 107
  Primer

SEQ ID NO: 151 - DNA, 70 bp, Artificial Sequence; identical to SEQ ID NO: 108
  Primer

SEQ ID NO: 152 - DNA, 70 bp, Artificial Sequence; identical to SEQ ID NO: 109
  Primer

SEQ ID NO: 153 - DNA, 70 bp, Artificial Sequence; identical to SEQ ID NO: 110
  Primer

SEQ ID NO: 154 - DNA, 70 bp, Artificial Sequence; identical to SEQ ID NO: 111
  Primer

SEQ ID NO: 155 - DNA, 70 bp, Artificial Sequence; identical to SEQ ID NO: 112
  Primer

SEQ ID NO: 156 - DNA, 26 bp, Artificial Sequence
  Primer
cacgtctcccaaatgcttgagtgacg

SEQ ID NO: 157 - DNA, 20 bp, Artificial Sequence
  Primer
cctcgaggccagggcgttcc

SEQ ID NO: 158 - DNA, 42 bp, Artificial Sequence; identical to SEQ ID NO: 3
  Primer

SEQ ID NO: 159 - DNA, 43 bp, Artificial Sequence; identical to SEQ ID NO: 4
  Primer

SEQ ID NO: 160 - DNA, 38 bp, Artificial Sequence; identical to SEQ ID NO: 5
  Primer

SEQ ID NO: 161 - DNA, 41 bp, Artificial Sequence; identical to SEQ ID NO: 6
  Primer

SEQ ID NO: 162 - DNA, 42 bp, Artificial Sequence; identical to SEQ ID NO: 7
  Primer

SEQ ID NO: 163 - DNA, 43 bp, Artificial Sequence; identical to SEQ ID NO: 4
  Primer

SEQ ID NO: 164 - DNA, 23 bp, Artificial Sequence
  Primer
tcggggaccaagacctaatcaga

SEQ ID NO: 165 - DNA, 23 bp, Artificial Sequence
  Primer
agccgcaggaagaagagtcacag

SEQ ID NO: 166 - DNA, 19 bp, Artificial Sequence
  Primer
aggccagggcgttccaatc

SEQ ID NO: 167 - DNA, 21 bp, Artificial Sequence
  Primer
caatagcgcgagggcagtttc

SEQ ID NO: 168 - DNA, 23 bp, Artificial Sequence
  Primer
ggcacccgcaatcctaataacaa

SEQ ID NO: 169 - DNA, 23 bp, Artificial Sequence
  Primer
agccgcaggaagaagagtcacag

SEQ ID NO: 170 - DNA, 28 bp, Artificial Sequence
  Primer
acatcacagcttctacacccgttaaggt

SEQ ID NO: 171 - DNA, 28 bp, Artificial Sequence
  Primer
atacagaatacatagattgctgttatcc

SEQ ID NO: 172 - DNA, 27 bp, Artificial Sequence
  Primer
gcatcgttgactatggtgtccgattct

SEQ ID NO: 173 - DNA, 28 bp, Artificial Sequence
  Primer
gctgcattggtttgttatatcgttatgc

SEQ ID NO: 174 - DNA, 42 bp, Artificial Sequence; identical to SEQ ID NO: 19
  Primer

SEQ ID NO: 175 - DNA, 43 bp, Artificial Sequence; identical to SEQ ID NO: 20
  Primer

SEQ ID NO: 176 - DNA, 43 bp, Artificial Sequence; identical to SEQ ID NO: 21
  Primer

SEQ ID NO: 177 - DNA, 43 bp, Artificial Sequence; identical to SEQ ID NO: 22
  Primer

SEQ ID NO: 178 - DNA, 41 bp, Artificial Sequence; identical to SEQ ID NO: 23
  Primer

SEQ ID NO: 179 - DNA, 44 bp, Artificial Sequence; identical to SEQ ID NO: 24
  Primer

SEQ ID NO: 180 - DNA, 43 bp, Artificial Sequence; identical to SEQ ID NO: 25
  Primer

SEQ ID NO: 181 - DNA, 43 bp, Artificial Sequence; identical to SEQ ID NO: 26
  Primer

SEQ ID NO: 182 - DNA, 43 bp, Artificial Sequence; identical to SEQ ID NO: 27
  Primer

SEQ ID NO: 183 - DNA, 43 bp, Artificial Sequence; identical to SEQ ID NO: 28
  Primer

SEQ ID NO: 184 - DNA, 42 bp, Artificial Sequence; identical to SEQ ID NO: 29
  Primer

SEQ ID NO: 185 - DNA, 44 bp, Artificial Sequence; identical to SEQ ID NO: 30
  Primer

SEQ ID NO: 186 - DNA, 43 bp, Artificial Sequence; identical to SEQ ID NO: 31
  Primer

SEQ ID NO: 187 - DNA, 43 bp, Artificial Sequence; identical to SEQ ID NO: 32
  Primer

SEQ ID NO: 188 - DNA, 42 bp, Artificial Sequence; identical to SEQ ID NO: 33
  Primer

SEQ ID NO: 189 - DNA, 44 bp, Artificial Sequence; identical to SEQ ID NO: 34
  Primer

SEQ ID NO: 190 - DNA, 23 bp, Artificial Sequence
  Primer
agccgcttgtcacaatgccaatt

SEQ ID NO: 191 - DNA, 23 bp, Artificial Sequence
  Primer
catcaccaagctcgccaacagtt

SEQ ID NO: 192 - DNA, 24 bp, Artificial Sequence
  Primer
aggttgccatcattttggcatctt

SEQ ID NO: 193 - DNA, 23 bp, Artificial Sequence
  Primer
ctttgcgccagcgatagtgactt

SEQ ID NO: 194 - DNA, 22 bp, Artificial Sequence
  Primer
atggcacccgtttctgcaatgg

SEQ ID NO: 195 - DNA, 24 bp, Artificial Sequence
  Primer
tcgggcagctgacacgaatgtaga

SEQ ID NO: 196 - DNA, 24 bp, Artificial Sequence
  Primer
gaatggcgatgtagtggctattga

SEQ ID NO: 197 - DNA, 23 bp, Artificial Sequence
  Primer
taatgccggcatccaaacataat

SEQ ID NO: 198 - DNA, 24 bp, Artificial Sequence
  Primer
tagccagcgtggtggttcatacaa

SEQ ID NO: 199 - DNA, 23 bp, Artificial Sequence
  Primer
ctcccggcagaaagctgtaagct

SEQ ID NO: 200 - DNA, 23 bp, Artificial Sequence
  Primer
tatagagcccgtgctggtgatgc

SEQ ID NO: 201 - DNA, 24 bp, Artificial Sequence
  Primer
atcgccattcaagtctgggaagaa

SEQ ID NO: 202 - DNA, 24 bp, Artificial Sequence
  Primer
tggctcaggccatactggcattac

SEQ ID NO: 203 - DNA, 23 bp, Artificial Sequence
  Primer
tttgcgccagcgatagtgacttg

SEQ ID NO: 204 - DNA, 23 bp, Artificial Sequence
  Primer
ttcccgtcaggcaaagttgaagg

SEQ ID NO: 205 - DNA, 24 bp, Artificial Sequence
  Primer
gacggcaattcctgtttgagcaga

SEQ ID NO: 206 - DNA, 43 bp, Artificial Sequence
  Primer
tcacttgcttccgttgaggatgaattaccaagtcaatggttac

SEQ ID NO: 207 - DNA, 41 bp, Artificial Sequence
  Primer
ggtttcggatgttacagcgtataaccagtcggtacagctac

SEQ ID NO: 208 - DNA, 39 bp, Artificial Sequence
  Primer
tcacttgcttccgttgagggaagctattcgtcacgttcg

SEQ ID NO: 209 - DNA, 42 bp, Artificial Sequence
  Primer
ggtttcggatgttacagcgtctgtagaaaatcctagctggag

SEQ ID NO: 210 - DNA, 40 bp, Artificial Sequence
  Primer
tcacttgcttccgttgaggcctctcttgttcttgctcgca

SEQ ID NO: 211 - DNA, 37 bp, Artificial Sequence
  Primer
ggtttcggatgttacagcgtgtgagccgccacacatg

SEQ ID NO: 212 - DNA, 40 bp, Artificial Sequence
  Primer
tcacttgcttccgttgaggctaacatgcttaggataatgg

SEQ ID NO: 213 - DNA, 41 bp, Artificial Sequence
  Primer
ggtttcggatgttacagcgtcaggtaagcgtaaaactcatc

SEQ ID NO: 214 - DNA, 40 bp, Artificial Sequence
  Primer
tcacttgcttccgttgagggcctctcttgttcttgctcgc

SEQ ID NO: 215 - DNA, 45 bp, Artificial Sequence
  Primer
tcacttgcttccgttgaggcaccgtttctacaggttagctaacga

SEQ ID NO: 216 - DNA, 46 bp, Artificial Sequence
  Primer
ggtttcggatgttacagcgtaaatgtttacgcaggtaagcgtaaaa

SEQ ID NO: 217 - DNA, 36 bp, Artificial Sequence
  Primer
tcacttgcttccgttgaggtacacacctcagcgttg

SEQ ID NO: 218 - DNA, 36 bp, Artificial Sequence
  Primer
ggtttcggatgttacagcgtcacgaacgtgacgaat

SEQ ID NO: 219 - DNA, 39 bp, Artificial Sequence
  Primer
tcacttgcttccgttgagggcttaggataatggcctctc

SEQ ID NO: 220 - DNA, 44 bp, Artificial Sequence
  Primer
ggtttcggatgttacagcgtccacgaattcatgatcaacatccc

SEQ ID NO: 221 - DNA, 40 bp, Artificial Sequence
  Primer
tcacttgcttccgttgagggctcgcaaacataacacttgc

SEQ ID NO: 222 - DNA, 41 bp, Artificial Sequence
  Primer
ggtttcggatgttacagcgtgagacactcatagagcctgtg

SEQ ID NO: 223 - DNA, 24 bp, Artificial Sequence
  Primer
atgaattaccaagtcaatggttac

SEQ ID NO: 224 - DNA, 21 bp, Artificial Sequence
  Primer
ataaccagtcggtacagctac

SEQ ID NO: 225 - DNA, 20 bp, Artificial Sequence
  Primer
gaagctattcgtcacgttcg

SEQ ID NO: 226 - DNA, 22 bp, Artificial Sequence
  Primer
ctgtagaaaatcctagctggag

SEQ ID NO: 227 - DNA, 21 bp, Artificial Sequence
  Primer
cctctcttgttcttgctcgca

SEQ ID NO: 228 - DNA, 17 bp, Artificial Sequence
  Primer
gtgagccgccacacatg

SEQ ID NO: 229 - DNA, 21 bp, Artificial Sequence
  Primer
ctaacatgcttaggataatgg

SEQ ID NO: 230 - DNA, 21 bp, Artificial Sequence
  Primer
caggtaagcgtaaaactcatc

SEQ ID NO: 231 - DNA, 21 bp, Artificial Sequence
  Primer
gcctctcttgttcttgctcgc

SEQ ID NO: 232 - DNA, 26 bp, Artificial Sequence
  Primer
caccgtttctacaggttagctaacga

SEQ ID NO: 233 - DNA, 26 bp, Artificial Sequence
  Primer
aaatgtttacgcaggtaagcgtaaaa

SEQ ID NO: 234 - DNA, 17 bp, Artificial Sequence
  Primer
tacacacctcagcgttg

SEQ ID NO: 235 - DNA, 16 bp, Artificial Sequence
  Primer
cacgaacgtgacgaat

SEQ ID NO: 236 - DNA, 20 bp, Artificial Sequence
  Primer
gcttaggataatggcctctc

SEQ ID NO: 237 - DNA, 24 bp, Artificial Sequence
  Primer
ccacgaattcatgatcaacatccc

SEQ ID NO: 238 - DNA, 21 bp, Artificial Sequence
  Primer
gctcgcaaacataacacttgc

SEQ ID NO: 239 - DNA, 21 bp, Artificial Sequence
  Primer
gagacactcatagagcctgtg

SEQ ID NO: 240 - DNA, 26 bp, Artificial Sequence
  Primer
ccagctccaataggaatgtcgcactc

SEQ ID NO: 241 - DNA, 28 bp, Artificial Sequence
  Primer
tccgcagatgtacatattacaatctacg

SEQ ID NO: 242 - DNA, 23 bp, Artificial Sequence
  Primer
ttaaatgcaccggccacggtttg

SEQ ID NO: 243 - DNA, 24 bp, Artificial Sequence
  Primer
atagcgccaggacaaactggtgtt

SEQ ID NO: 244 - DNA, 24 bp, Artificial Sequence
  Primer
tatatgcgccaagctggtgtgagt

SEQ ID NO: 245 - DNA, 24 bp, Artificial Sequence
  Primer
cgaggcggaggtacaaattgacag

SEQ ID NO: 246 - DNA, 24 bp, Artificial Sequence
  Primer
atgaagccgagccaaacataccaa

SEQ ID NO: 247 - DNA, 40 bp, Artificial Sequence; identical to SEQ ID NO: 58
  Primer

SEQ ID NO: 248 - DNA, 44 bp, Artificial Sequence; identical to SEQ ID NO: 59
  Primer

SEQ ID NO: 249 - DNA, 43 bp, Artificial Sequence; identical to SEQ ID NO: 60
  Primer

SEQ ID NO: 250 - DNA, 40 bp, Artificial Sequence; identical to SEQ ID NO: 61
  Primer

SEQ ID NO: 251 - DNA, 43 bp, Artificial Sequence; identical to SEQ ID NO: 62
  Primer

SEQ ID NO: 252 - DNA, 44 bp, Artificial Sequence; identical to SEQ ID NO: 63
  Primer

SEQ ID NO: 253 - DNA, 43 bp, Artificial Sequence; identical to SEQ ID NO: 64
  Primer

SEQ ID NO: 254 - DNA, 44 bp, Artificial Sequence; identical to SEQ ID NO: 65
  Primer

SEQ ID NO: 255 - DNA, 21 bp, Artificial Sequence
  Primer
atgcaccggccacggtttgtg

SEQ ID NO: 256 - DNA, 24 bp, Artificial Sequence
  Primer
atgcgccaagctggtgtgagttga

SEQ ID NO: 257 - DNA, 24 bp, Artificial Sequence
  Primer
tgctggcgctgctcttcaaatacc

SEQ ID NO: 258 - DNA, 20 bp, Artificial Sequence
  Primer
cggggctgcttgtgggaagg

(19) United States
(12) Patent Application Publication
(10) Pub. No.: US 2007/0037140 A1
(43) Pub. Date: Feb. 15, 2007

(54) Methods and compositions for detecting sars virus

(75) Inventors: Ze Li, Beijing (CN); Yiming Zhou, Beijing (CN); Shengce Tao, Beijing (CN); Jing Cheng, Beijing (CN); Jianwei Wang, Beijing (CN); Tao Hong, Beijing (CN); Yun Cheng, Beijing (CN); Quanzhong Shan, Beijing (CN)

Correspondence Address: MORRISON & FOERSTER LLP, 12531 HIGH BLUFF DRIVE, SUITE 100, SAN DIEGO, CA, 92130-2040, US

(73) Assignees: CAPITAL BIOCHIP COMPANY, LTD., Beijing (CN); TSINGHUA UNIVERSITY, Beijing (CN)

(21) Appl. No.: 10/556,182

(22) PCT Filed: May 9, 2003
(86) PCT No.: PCT/CN03/00336

§ 371 Date: Aug. 22, 2006

(51) Int. Cl. C12Q 1/70; C12Q 1/68; C12M 1/34

(52) U.S. Cl. 435/5; 435/287.2; 435/6; 977/924

(57) ABSTRACT

This invention relates generally to the field of virus detection. In particular, the invention provides chips, probes, primers, kits and methods for amplifying and detecting SARS-CoV nucleotide sequence. The clinical and other uses of the present chips, probes, primers, kits and methods are also contemplated.

### BACKGROUND OF THE INVENTION

Since November of 2002, a disease called severe acute respiratory syndrome (SARS) has been reported in twenty two countries around world. WHO has reported 6,054 cumulative cases of SARS and 417 death among infected people as of May 2, 2003. For the same period, China has reported 3,788 cumulative cases of SARS and 181 death among infected people.

The main symptoms for SARS patients include fever (greater than 38° C.), headache, body aches. After 2-7 days of illness, patients may develop a dry, nonproductive cough that may be accompanied with breathing difficulty.

Based on findings from Hong Kong, Canada, and U.S., a previously unrecognized coronaviruse has been identified as the cause of SARS. Researchers have found that SARS coronaviruse is a positive chain RNA virus which replicates without DNA intermediate step and uses standard codon (Marra et al., Science 2003 May 1; (epub ahead of print); and Rota et al., Science 2003 May 1, (epub ahead of print)).

SARS coronaviruse is a newly discovered virus which has not been previously detected in human or animals. The genome structure of SARS coronaviruse is very similar to other coronaviruse. The genome of SARS coronaviruse is 30 K base pairs in length and the genome is considered very large for a virus. The genome of SARS coronaviruse encodes RNA polymerase (polymerase 1a and 1b), S protein (spike protein), M protein (membrane protein), and N protein (nucleocapsid protein), etc.

Currently, there are three types of detection methods for SARS coronaviruse: immunological methods (e.g., ELISA), reverse transcriptase polymerase chain reaction (RT-PCR) tests, and cell culture methods.

There are significant drawbacks of the above three detection methods. For example, ELISA can reliably detect antibodies from serum of SARS patients. However, those antibodies can only be detected twenty one days after development of symptoms. Cell culture methods have a relative long detection cycle and can be applied only to limited conditions. In addition, cell culture methods can only detect existence of alive virus.

The key step of preventing the spread of SARS coronaviruse is early diagnosis and early quarantine and treatment. RT-PCR is the only existing method that allows detection of nucleic acid of SARS coronaviruse. However, RT-PCR cannot eliminate infected patient before SARS virus expression, and detection rate for RT-PCR is low. The detection process requires expensive real time PCR equipment. Thus, RT-PCR cannot satisfy the need of early clinical screening and diagnosis.

There exists a need in the art for a quick, sensitive and accurate diagnosis of the severe acute respiratory syndrome (SARS). The present invention address this and other related needs in the art.

### BRIEF SUMMARY OF THE INVENTION

In one aspect, the present invention is directed to a chip for assaying for a coronaviruse causing the severe acute respiratory syndrome (SARS-CoV), which chip comprises a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotides.

In another aspect, the present invention is directed to a method for assaying for a SARS-CoV in a sample, which methods comprises: a) providing an above-described chip; b) contacting said chip with a sample containing or suspected of containing a SARS-CoV nucleotide sequence under conditions suitable for nucleic acid hybridization; and c) assessing hybrids formed between said SARS-CoV nucleotide sequence, if present in said sample, and said at least two oligonucleotide probes complementary to two different nucleotide sequences of SARS-CoV genome, respectively, to determine the presence, absence or amount of said SARS-CoV in said sample, whereby detection of one or both said hybrids indicates the presence of said SARS-CoV in said sample.

By using multiple hybridization probes, the present methods reduce the occurrence of false negative results compared to a test based on a single hybridization probe as the chance of simultaneous mutations of the multiple hybridization targets is much smaller than the chance of a mutation in the single hybridization target. When other preferred embodiments are used, e.g., a negative control probe and a blank spot on the chip, the chance of a false positive result can also be reduced. The inclusion of more preferred embodiments, e.g., an immobilization control probe and a positive control probe, on the chip can provide further validation of the assay results. The use of preferred sample preparation procedures, RNA extraction procedures and amplification procedures can further enhance the sensitivity of the present methods.

In still another aspect, the present invention is directed to an oligonucleotide primer for amplifying a SARS-CoV nucleotide sequence, which oligonucleotide primer comprises a nucleotide sequence that: a) hybridizes, under high stringency, with a target SARS-CoV nucleotide sequence, or a complementary strand thereof, that is set forth in Table 1; or b) has at least 90% identity to a target SARS-CoV nucleotide sequence comprising a nucleotide sequence, or a complementary strand thereof, that is set forth in Table 1. The kits and methods for amplifying a SARS-CoV nucleotide sequence using the primers are also contemplated.

TABLE 1
SARS-CoV primers designed by Capital Biochip

| | | |
|---|---|---|
| id | sequence (5′-3′) | region |

| | | |
|---|---|---|
| PMSL_00005 | CACGTCTCCCAAATGCTTGAGTGACG | SARS-Cov Nucleocapsid gene |
| PMSU_00006 | CCTCGAGGCCAGGGCGTTCC | SARS-Cov Nucleocapsid gene |
| PMV_00039 | TCACTTGCTTCCGTTGAGGTCGGGGACCAAGACCTAATCAGA | SARS-Cov Nucleocapsid gene |
| PMV_00040 | GGTTTCGGATGTTACAGCGTAGCCGCAGGAAGAAGAGTCACAG | SARS-Cov Nucleocapsid gene |
| PMV_00041 | TCACTTGCTTCCGTTGAGGAGGCCAGGGCGTTCCAATC | SARS-Cov Nucleocapsid gene |
| PMV_00042 | GGTTTCGGATGTTACAGCGTCAATAGCGCGAGGGCAGTTTC | SARS-Cov Nucleocapsid gene |
| PMV_00043 | TCACTTGCTTCCGTTGAGGGGCACCCGCAATCCTAATAACAA | SARS-Cov Nucleocapsid gene |
| PMV_00044 | GGTTTCGGATGTTACAGCGTAGCCGCAGGAAGAAGAGTCACAG | SARS-Cov Nucleocapsid gene |
| PMV_00090 | TCGGGGACCAAGACCTAATCAGA | SARS-Cov Nucleocapsid gene |
| PMV_00091 | AGCCGCAGGAAGAAGAGTCACAG | SARS-Cov Nucleocapsid gene |
| PMV_00092 | AGGCCAGGGCGTTCCAATC | SARS-Cov Nucleocapsid gene |
| PMV_00093 | CAATAGCGCGAGGGCAGTTTC | SARS-Cov Nucleocapsid gene |
| PMV_00094 | GGCACCCGCAATCCTAATAACAA | SARS-Cov Nucleocapsid gene |
| PMV_00095 | AGCCGCAGGAAGAAGAGTCACAG | SARS-Cov Nucleocapsid gene |
| PMSL_00001 | ACATCACAGCTTCTACACCCGTTAAGGT | SARS-Cov Replicase 1A |
| PMSL_00002 | ATACAGAATACATAGATTGCTGTTATCC | SARS-Cov Replicase 1A |
| PMSU_00002 | GCATCGTTGACTATGGTGTCCGATTCT | SARS-Cov Replicase 1A |
| PMSU_00003 | GCTGCATTGGTTTGTTATATCGTTATGC | SARS-Cov Replicase 1A |
| PMV_00023 | TCACTTGCTTCCGTTGAGGAGCCGCTTGTCACAATGCCAATT | SARS-Cov Replicase 1A |
| PMV_00024 | GGTTTCGGATGTTACAGCGTCATCACCAAGCTCGCCAACAGTT | SARS-Cov Replicase 1A |
| PMV_00025 | TCACTTGCTTCCGTTGAGGAGGTTGCCATCATTTTGGCATCTT | SARS-Cov Replicase 1A |
| PMV_00026 | GGTTTCGGATGTTACAGCGTCTTTGCGCCAGCGATAGTGACTT | SARS-Cov Replicase 1A |
| PMV_00027 | TCACTTGCTTCCGTTGAGGATGGCACCCGTTTCTGCAATGG | SARS-Cov Replicase 1A |
| PMV_00028 | GGTTTCGGATGTTACAGCGTTCGGGCAGCTGACACGAATGTAGA | SARS-Cov Replicase 1A |
| PMV_00029 | TCACTTGCTTCCGTTGAGGGAATGGCGATGTAGTGGCTATTGA | SARS-Cov Replicase 1A |
| PMV_00030 | GGTTTCGGATGTTACAGCGTTAATGCCGGCATCCAAACATAAT | SARS-Cov Replicase 1A |
| PMV_00031 | TCACTTGCTTCCGTTGAGGTAGCCAGCGTGGTGGTTCATACAA | SARS-Cov Replicase 1A |
| PMV_00032 | GGTTTCGGATGTTACAGCGTCTCCCGGCAGAAAGCTGTAAGCT | SARS-Cov Replicase 1A |
| PMV_00033 | TCACTTGCTTCCGTTGAGGTATAGAGCCCGTGCTGGTGATGC | SARS-Cov Replicase 1A |
| PMV_00034 | GGTTTCGGATGTTACAGCGTATCGCCATTCAAGTCTGGGAAGAA | SARS-Cov Replicase 1A |
| PMV_00035 | TCACTTGCTTCCGTTGAGGTGGCTCAGGCCATACTGGCATTAC | SARS-Cov Replicase 1A |
| PMV_00036 | GGTTTCGGATGTTACAGCGTTTTGCGCCAGCGATAGTGACTTG | SARS-Cov Replicase 1A |
| PMV_00037 | TCACTTGCTTCCGTTGAGGTTCCCGTCAGGCAAAGTTGAAGG | SARS-Cov Replicase 1A |
| PMV_00038 | GGTTTCGGATGTTACAGCGTGACGGCAATTCCTGTTTGAGCAGA | SARS-Cov Replicase 1A |
| PMV_00074 | AGCCGCTTGTCACAATGCCAATT | SARS-Cov Replicase 1A |
| PMV_00075 | CATCACCAAGCTCGCCAACAGTT | SARS-Cov Replicase 1A |
| PMV_00076 | AGGTTGCCATCATTTTGGCATCTT | SARS-Cov Replicase 1A |
| PMV_00077 | CTTTGCGCCAGCGATAGTGACTT | SARS-Cov Replicase 1A |
| PMV_00078 | ATGGCACCCGTTTCTGCAATGG | SARS-Cov Replicase 1A |
| PMV_00079 | TCGGGCAGCTGACACGAATGTAGA | SARS-Cov Replicase 1A |
| PMV_00080 | GAATGGCGATGTAGTGGCTATTGA | SARS-Cov Replicase 1A |
| PMV_00081 | TAATGCCGGCATCCAAACATAAT | SARS-Cov Replicase 1A |
| PMV_00082 | TAGCCAGCGTGGTGGTTCATACAA | SARS-Cov Replicase 1A |
| PMV_00083 | CTCCCGGCAGAAAGCTGTAAGCT | SARS-Cov Replicase 1A |
| PMV_00084 | TATAGAGCCCGTGCTGGTGATGC | SARS-Cov Replicase 1A |
| PMV_00085 | ATCGCCATTCAAGTCTGGGAAGAA | SARS-Cov Replicase 1A |
| PMV_00086 | TGGCTCAGGCCATACTGGCATTAC | SARS-Cov Replicase 1A |
| PMV_00087 | TTTGCGCCAGCGATAGTGACTTG | SARS-Cov Replicase 1A |
| PMV_00088 | TTCCCGTCAGGCAAAGTTGAAGG | SARS-Cov Replicase 1A |
| PMV_00089 | GACGGCAATTCCTGTTTGAGCAGA | SARS-Cov Replicase 1A |
| PMSL_00003 | CCAGCTCCAATAGGAATGTCGCACTC | SARS-Cov Spike glycoprotein gene |
| PMSL_00004 | TCCGCAGATGTACATATTACAATCTACG | SARS-Cov Spike glycoprotein gene |
| PMSU_00005 | TTAAATGCACCGGCCACGGTTTG | SARS-Cov Spike glycoprotein gene |
| PMV_000100 | ATAGCGCCAGGACAAACTGGTGTT | SARS-Cov Spike glycoprotein gene |
| PMV_000101 | TATATGCGCCAAGCTGGTGTGAGT | SARS-Cov Spike glycoprotein gene |
| PMV_000102 | CGAGGCGGAGGTACAAATTGACAG | SARS-Cov Spike glycoprotein gene |
| PMV_000103 | ATGAAGCCGAGCCAAACATACCAA | SARS-Cov Spike glycoprotein gene |
| PMV_00045 | TCACTTGCTTCCGTTGAGGATGCACCGGCCACGGTTTGTG | SARS-Cov Spike glycoprotein gene |
| PMV_00046 | GGTTTCGGATGTTACAGCGTATGCGCCAAGCTGGTGTGAGTTGA | SARS-Cov Spike glycoprotein gene |
| PMV_00047 | TCACTTGCTTCCGTTGAGGTGCTGGCGCTGCTCTTCAAATACC | SARS-Cov Spike glycoprotein gene |
| PMV_00048 | GGTTTCGGATGTTACAGCGTCGGGGCTGCTTGTGGGAAGG | SARS-Cov Spike glycoprotein gene |
| PMV_00049 | TCACTTGCTTCCGTTGAGGATAGCGCCAGGACAAACTGGTGTT | SARS-Cov Spike glycoprotein gene |
| PMV_00050 | GGTTTCGGATGTTACAGCGTTATATGCGCCAAGCTGGTGTGAGT | SARS-Cov Spike glycoprotein gene |
| PMV_00051 | TCACTTGCTTCCGTTGAGGCGAGGCGGAGGTACAAATTGACAG | SARS-Cov Spike glycoprotein gene |
| PMV_00052 | GGTTTCGGATGTTACAGCGTATGAAGCCGAGCCAAACATACCAA | SARS-Cov Spike glycoprotein gene |
| PMV_00096 | ATGCACCGGCCACGGTTTGTG | SARS-Cov Spike glycoprotein gene |
| PMV_00097 | ATGCGCCAAGCTGGTGTGAGTTGA | SARS-Cov Spike glycoprotein gene |
| PMV_00098 | TGCTGGCGCTGCTCTTCAAATACC | SARS-Cov Spike glycoprotein gene |
| PMV_00099 | CGGGGCTGCTTGTGGGAAGG | SARS-Cov Spike glycoprotein gene |

In yet another aspect, the present invention is directed to an oligonucleotide probe for hybridizing to a SARS-CoV nucleotide sequence, which oligonucleotide probe comprises a nucleotide sequence that: a) hybridizes, under high stringency, with a target SARS-CoV nucleotide sequence, or a complementary strand thereof, that is set forth in Table 2; or b) has at least 90% identity to a target SARS-CoV nucleotide sequence comprising a nucleotide sequence, or a complementary strand thereof, that is set forth in Table 2. The kits and methods for hybridization analysis of a SARS-CoV nucleotide sequence using the probes are also contemplated.

TABLE 2

| | | |
|---|---|---|
| probe_id | sequence | region |

| | | |
|---|---|---|
| PBS00001 | TTACCCTAATATGTTTATCACCCGCGAAGAAGCTATTCGTCACGTTCGTGCGTGGA | SARS-Cov Replicase 1B |
| PBS00002 | CTGACAAGTATGTCCGCAATCTACAACACAGGCTCTATGAGTGTCTCTATAGAAAT | SARS-Cov Replicase 1B |
| PBS00003 | CATAACACTTGCTGTAACTTATCACACCGTTTCTACAGGTTAGCTAACGAGTGTGC | SARS-Cov Replicase 1B |
| PBS00004 | TTACCCTAATATGTTTATCACCCGCGAAGAAGCTATTCGTCACGTTCGT | SARS-Cov Replicase 1B |
| PBS00009 | GCGTTCTCTTAAAGCTCCTGCCGTAGTGTCAGTATCATCACCAGATGCTGTTACT | SARS-Cov Replicase 1A |
| | ACATATAATGGATAC | |
| PBS00010 | CTTTGGCTGGCTCTTACAGAGATTGGTCCTATTCAGGACAGCGTACAGAGTTAGG | SARS-Cov Replicase 1A |
| | TGTTGAATTTCTTAA | |
| PBS00011 | CTACGTAGTGAAGCTTTCGAGTACTACCATACTCTTGATGAGAGTTTTCTTGGTA | SARS-Cov Replicase 1A |
| | GGTACATGTCTGCTT | |
| PBS00012 | TGCCAATTGGTTATGTGACACATGGTTTTAATCTTGAAGAGGCTGCGCGCTGTAT | SARS-Cov Replicase 1A |
| | GCGTTCTCTTAAAGC | |
| PBS00013 | TATAAAGTTACCAAGGGAAAGCCCGTAAAAGGTGCTTGGAACATTGGACAACAGA | SARS-Cov Replicase 1A |
| | GATCAGTTTTAACAC | |
| PBS00014 | TGCTTCATTGATGTTGTTAACAAGGCACTCGAAATGTGCATTGATCAAGTCACTA | SARS-Cov Replicase 1A |
| | TCGCTGGCGCAAAG | |
| PBS00015 | TGTCGACGCCATGGTTTATACTTCAGACCTGCTCACCAACAGTGTCATTATTATG | SARS-Cov Replicase 1A |
| | GCATATGTAACTGGT | |
| PBS00016 | TACTGTTGAAAAACTCAGGCCTATCTTTGAATGGATTGAGGCGAAACTTAGTGCA | SARS-Cov Replicase 1A |
| | GGAGTTGAATTTCTC | |
| PBS00017 | ACCTATTCTGTTGCTTGACCAAGCTCTTGTATCAGACGTTGGAGATAGTACTGAA | SARS-Cov Replicase 1A |
| | GTTTCC | |
| PBS00018 | GCCTATTAATGTCATAGTTTTTGATGGCAAGTCCAAATGCGACGAGTCTGCTTCT | SARS-Cov Replicase 1A |
| | AAGTCTGCTTCTGTG | |
| PBS00019 | TGAGAGCTAACAACACTAAAGGTTCACTGCCTATTAATGTCATAGTTTTTGATGG | SARS-Cov Replicase 1A |
| | CAAGTCCAAATGCGA | |
| PBS00020 | ACTTGCATGATGTGCTATAAGCGCAATCGTGCCACACGCGTTGAGTGTACAACTA | SARS-Cov Replicase 1A |
| | TTGTTAATGGCATGA | |
| PBS00021 | GGCGATGTAGTGGCTATTGACTATAGACACTATTCAGCGAGTTTCAAGAAAGGTG | SARS-Cov Replicase 1A |
| | CTAAATTACTGCATA | |
| PBS00022 | TCAAACCAAACACTTGGTGTTTACGTTGTCTTTGGAGTACAAAGCCAGTAGATAC | SARS-Cov Replicase 1A |
| | TTCAAATTCATTTGA | |
| PBS00023 | TAGTGCTGTTGGCAACATTTGCTACACACCTTCCAAACTCATTGAGTATAGTGAT | SARS-Cov Replicase 1A |
| | TTTGCTAC | |
| PBS00024 | TCATAGCTAACATCTTTACTCCTCTTGTGCAACCTGTGGGTGCTTTAGATGTGTC | SARS-Cov Replicase 1A |
| | TGCTTCAGTAGTGGC | |
| PBS00025 | GGTATTATTGCCATATTGGTGACTTGTGCTGCCTACTACTTTATGAAATTCAGAC | SARS-Cov Replicase 1A |
| | GTGTTTTTGGTGAGT | |
| PBS00026 | GTGATGTCAGAGAAACTATGACCCATCTTCTACAGCATGCTAATTTGGAATCTGC | SARS-Cov Replicase 1A |
| | AAAGCGAGTTCTTAA | |
| PBS00027 | AACCATCAAGCCTGTGTCGTATAAACTCGATGGAGTTACTTACACAGAGATTGAA | SARS-Cov Replicase 1A |
| | CCAAAATTGGATGGG | |
| PBS00028 | GTTTTCTACAAGGAAACATCTTACACTACAACCATCAAGCCTGTGTCGTATAAAC | SARS-Cov Replicase 1A |
| | TCGATGGAGTTACTT | |
| PBS00029 | CCTTGAATGAGGATCTCCTTGAGATACTGAGTCGTGAACGTGTTAACATTAACAT | SARS-Cov Replicase 1A |
| | TGTTGGCGATTTTCA | |
| PBS00031 | GCCATGGTTTATACTTCAGACCTGCTCACCAACAGTGTCATTATTATGGCATATG | SARS-Cov Replicase 1A |
| | TAACTGGTGGTCTTG | |
| PBS00032 | CAACAGACTTCTCAGTGGTTGTCTAATCTTTTGGGCACTACTGTTGAAAAACTCA | SARS-Cov Replicase 1A |
| | GGCCTATCTTTGAAT | |
| PBS00033 | TTCCCGTCAGGCAAAGTTGAAGGGTGCATGGTACAAGTAACCTGTGGAACTACAAC | SARS-Cov Replicase 1A |
| PBS00034 | GGTTCACCATCTGGTGTTTATCAGTGTGCCATGAGACCTAATCATACCATTAAAGG | SARS-Cov Replicase 1A |
| PBS00035 | AGATCATGTTGACATATTGGGACCTCTTTCTGCTCAAACAGGAATTGCCGTC | SARS-Cov Replicase 1A |
| PBS00036 | TAAAAAGGACAAAAAGAAAAAGACTGATGAAGCTCAGCCTTTGCCGCAGAGACAA | SARS-Cov Nucleocapsid gene |
| PBS00037 | ACGGCAAAATGAAAGAGCTCAGCCCCAGATGGTACTTCTATTACCTAGGAACTGG | SARS-Cov Nucleocapsid gene |
| | CCCAGAAGCTTCACT | |
| PBS00038 | GGCGCTAACAAAGAAGGCATCGTATGGGTTGCAACTGAGGGAGCCTTGAATACAC | SARS-Cov Nucleocapsid gene |
| | CCAAAGACCACATTG | |
| PBS00039 | GTCCAGATGACCAAATTGGCTACTACCGAAGAGCTACCCGACGAGTTCGTGGTGG | SARS-Cov Nucleocapsid gene |
| | TGACGGCAAAATGAA | |
| PBS00040 | GAGGTGGTGAAACTGCCCTCGCGCTATTGCTGCTAGACAGATTGAACCAGCTTGA | SARS-Cov Nucleocapsid gene |
| | GAGCAAAGTTTCTGG | |
| PBS00041 | AAAAGAAAAAGACTGATGAAGCTCAGCCTTTGCCGCAGAGACAAAAGAAGCAGCC | SARS-Cov Nucleocapsid gene |
| | CACTGTGACTCTTCT | |
| PBS00042 | AAATTGCACAATTTGCTCCAAGTGCCTCTGCATTCTTTGGAATGTCACGCATTGG | SARS-Cov Nucleocapsid gene |
| | CATGGAAGTCACACC | |
| PBS00043 | ACCAATTTAACAAGGCGATTAGTCAAATTCAAGAATCACTTACAACAACATCAAC | SARS-Cov Spike glycoprotein gene |
| | TGCATTGGGCAAGCT | |
| PBS00044 | CACCTGGAACAAATGCTTCATCTGAAGTTGCTGTTCTATATCAAGATGTTAACTG | SARS-Cov Spike glycoprotein gene |
| | CACTGATGTTTCTAC | |
| PBS00045 | AAAGGGCTACCACCTTATGTCCTTCCCACAAGCAGCCCCGCATGGTGTTGTCTTC | SARS-Cov Spike glycoprotein gene |
| | CTACATGTCACGTAT | |
| PBS00046 | TCAGGAAATTGTGATGTCGTTATTGGCATCATTAACAACACAGTTTATGATCCTC | SARS-Cov Spike glycoprotein gene |
| | TGCAACCTGAGCTTG | |
| PBS00047 | TTGATCTTGGCGACATTTCAGGCATTAACGCTTCTGTCGTCAACATTCAAAAAGA | SARS-Cov Spike glycoprotein gene |
| | AATTGACCGCCTCAA | |
| PBS00048 | GAGGAACTTCACCACAGCGCCAGCAATTTGTCATGAAGGCAAAGCATACTTCCCT | SARS-Cov Spike glycoprotein gene |
| | CGTGAAGGTGTTTTT | |

### BRIEF DESCRIPTION OF THE SEVERAL VIEWS OF THE DRAWING(S)

FIGS. 1A and 1B illustrate exemplary SARS-CoV genome structures (See FIG. 2 of Marra et al., Science 2003 May 1; [epub ahead of print]; and GenBank Accession No. NC_{—}004718).

FIG. 2 illustrates an exemplary sample preparation procedure.

FIG. 3 illustrates an exemplary probe labeling to be used in PCR. The sequence of the universal primer is complementary to the common sequence of the specific primer. The universal primers and the specific primers are added into the PCR master mix before the amplification are performed. The specificity of the amplification is ensured by the specific part of the specific primer. After one or a few thermal cycles, the universal primer can be incorporated into the amplicon efficiently. Then the universal primer can anneal to the complementary sequence of the common sequence of the specific primer The PCR can further proceed with the fluorescence dye incorporated in the universal primer. 1 and 6 depict a fluorescence dye; 2 depicts an upstream universal primer; 3 depicts an upstream specific primer with a common sequence; 4 depicts a template; 5 depicts a downstream specific primer with a common sequence; and 7 depicts a downstream universal primer.

FIG. 4 illustrates probe immobilization on a glass slide surface modified with an amino group, e.g., poly-L-lysine treated. Amine Coupling Chemistry: Amine Substrates contain primary amine groups (NH3⁺) attached covalently to the glass surface (rectangles). The amines carry a positive charge at neutral pH, allowing attachment of natively charged DNA (double helix) through the formation of ionic bonds with the negatively charged phosphate backbone (middle panel). Electrostatic attachment is supplemented by treatment with an ultraviolet light or heat, which induces covalent attachment of the DNA to the surface through the covalent binding between the primary amine and thymine (right panel). The combination of electrostatic binding and covalent attachment couples the DNA to the substrate in a highly stable manner.

FIG. 5 illustrates an exemplary array format of SARS-CoV detection chip.

FIGS. 6A and 6B illustrate SARS-CoV detection from a SARS patient blood sample (sample No. 3).

FIGS. 7A and 7B illustrate SARS-CoV detection from a SARS patient blood sample (sample No. 4).

FIGS. 8A and 8B illustrate SARS-CoV detection from a SARS patient sputum sample (sample No. 5).

FIGS. 9A and 9B illustrate SARS-CoV detection from a SARS patient sputum sample (sample No. 6).

FIG. 10 illustrates another exemplary array format of SARS-CoV detection chip.

FIG. 11 illustrates all possible positive results on the SARS SARS-CoV detection chip illustrated in FIG. 10.

### DETAILED DESCRIPTION OF THE INVENTION

For clarity of disclosure, and not by way of limitation, the detailed description of the invention is divided into the subsections that follow.

### A. DEFINITIONS

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in the patents, applications, published applications and other publications that are herein incorporated by reference, the definition set forth in this section prevails over the definition that is incorporated herein by reference.

As used herein, “a” or “an” means “at least one” or “one or more.” As used herein, “coronaviridae” refers to a family of single-stranded RNA viruses responsible for respiratory diseases. The outer envelope of the virus has club-shaped projections that radiate outwards and give a characteristic corona appearance to negatively stained virions.

As used herein, “polymerase chain reaction (PCR)” refers to a system for in vitro amplification of DNA. Two synthetic oligonucleotide primers, which are complementary to two regions of the target DNA (one for each strand) to be amplified, are added to the target DNA (that need not be pure), in the presence of excess deoxynucleotides and a heat-stable DNA polymerase, e.g., Taq DNA polymerase. In a series, e.g., 30, of temperature cycles, the target DNA is repeatedly denatured (e.g., around 90° C.), annealed to the primers (e.g., at 50-60° C.) and a daughter strand extended from the primers (e.g., 72° C.). As the daughter strands themselves act as templates for subsequent cycles, DNA fragments matching both primers are amplified exponentially, rather than linearly. The original DNA need thus be neither pure nor abundant, and the PCR reaction has accordingly become widely used not only in research, but in clinical diagnostics and forensic science.

As used herein, “nested PCR” refers to a PCR in which specificity is improved by using two sets of primers sequentially. An initial PCR is performed with the “outer” primer pairs, then a small aliquot is used as a template for a second round of PCR with the “inner” primer pair.

As used herein, “reverse transcription PCR or RT-PCR” refers to PCR in which the starting template is RNA, implying the need for an initial reverse transcriptase step to make a DNA template. Some thermostable polymerases have appreciable reverse transcriptase activity; however, it is more common to perform an explicit reverse transcription, inactivate the reverse transcriptase or purify the product, and proceed to a separate conventional PCR.

As used herein, “primer” refers to an oligonucleotide that hybridizes to a target sequence, typically to prime the nucleic acid in the amplification process.

As used herein, “probe” refers to an oligonucleotide that hybridizes to a target sequence, typically to facilitate its detection. The term “target sequence” refers to a nucleic acid sequence to which the probe specifically binds. Unlike a primer that is used to prime the target nucleic acid in the amplification process, a probe need not be extended to amplify target sequence using a polymerase enzyme. However, it will be apparent to those skilled in the art that probes and primers are structurally similar or identical in many cases.

As used herein, “the concentration of said 5′ and 3′ universal primers equals to or is higher than the concentration of said 5′ and 3′ specific primers, respectively” means that the concentration of the 5′ universal primer equals to or is higher than the concentration of the 5′ specific primers and the concentration of the 3′ universal primer equals to or is higher than the concentration of the 3′ specific primers.

As used herein, “hairpin structure” refers to a polynucleotide or nucleic acid that contains a double-stranded stem segment and a single-stranded loop segment wherein the two polynucleotide or nucleic acid strands that form the double-stranded stem segment is linked and separated by the single polynucleotide or nucleic acid strand that forms the loop segment. The “hairpin structure” can further comprise 3′ and/or 5′ single-stranded region(s) extending from the double-stranded stem segment.

As used herein, “nucleic acid (s)” refers to deoxyribonucleic acid (DNA) and/or ribonucleic acid (RNA) in any form, including inter alia, single-stranded, duplex, triplex, linear and circular forms. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, “complementary or matched” means that two nucleic acid sequences have at least 50% sequence identity. Preferably, the two nucleic acid sequences have at least 60%, 70,%, 80%, 90%, 95%, 96%, 97%, 98%, 99% or 100% of sequence identity. “Complementary or matched” also means that two nucleic acid sequences can hybridize under low, middle and/or high stringency condition(s).

As used herein, “substantially complementary or substantially matched” means that two nucleic acid sequences have at least 90% sequence identity. Preferably, the two nucleic acid sequences have at least 95%, 96%, 97%, 98%, 99% or 100% of sequence identity. Alternatively, “substantially complementary or substantially matched” means that two nucleic acid sequences can hybridize under high stringency condition(s).

As used herein, “two perfectly matched nucleotide sequences” refers to a nucleic acid duplex wherein the two nucleotide strands match according to the Watson-Crick basepair principle, i.e., A-T and C-G pairs in DNA:DNA duplex and A-U and C-G pairs in DNA:RNA or RNA:RNA duplex, and there is no deletion or addition in each of the two strands.

As used herein: “stringency of hybridization” in determining percentage mismatch is as follows:

1) high stringency: 0.1×SSPE (or 0.1×SSC), 0.1% SDS, 65° C.;

2) medium stringency: 0.2×SSPE (or 1.0×SSC), 0.1% SDS, 50° C. (also referred to as moderate stringency); and

3) low stringency: 1.0×SSPE (or 5.0×SSC), 0.1% SDS, 50° C.

It is understood that equivalent stringencies may be achieved using alternative buffers, salts and temperatures.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

As used herein, “melting temperature” (“Tm”) refers to the midpoint of the temperature range over which nucleic acid duplex, i.e., DNA:DNA, DNA:RNA, RNA:RNA, PNA: DNA, LNA:RNA and LNA:DNA, etc., is denatured.

As used herein, “sample” refers to anything which may contain a target SARS-CoV to be assayed or amplified by the present chips, primers, probes, kits and methods. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s). Biological tissues may be processed to obtain cell suspension samples. The sample may also be a mixture of cells prepared in vitro. The sample may also be a cultured cell suspension. In case of the biological samples, the sample may be crude samples or processed samples that are obtained after various processing or preparation on the original samples. For example, various cell separation methods (e.g., magnetically activated cell sorting) may be applied to separate or enrich target cells from a body fluid sample such as blood. Samples used for the present invention include such target-cell enriched cell preparation.

As used herein, a “liquid (fluid) sample” refers to a sample that naturally exists as a liquid or fluid, e.g., a biological fluid. A “liquid sample” also refers to a sample that naturally exists in a non-liquid status, e.g., solid or gas, but is prepared as a liquid, fluid, solution or suspension containing the solid or gas sample material. For example, a liquid sample can encompass a liquid, fluid, solution or suspension containing a biological tissue.

As used herein, “assessing PCR products” refers to quantitative and/or qualitative determination of the PCR products, and also of obtaining an index, ratio, percentage, visual or other value indicative of the level of the PCR products. Assessment may be direct or indirect and the chemical species actually detected need not of course be the PCR products themselves but may, for example, be a derivative thereof, or some further substance.

### B. CHIPS FOR ASSAYING FOR A SARS-COV

In one aspect, the present invention is directed to a chip for assaying for a coronaviruse causing the severe acute respiratory syndrome (SARS-CoV), which chip comprises a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotides.

The at least two different nucleotide sequences can be any suitable combinations. For example, the at least two different nucleotide sequences of SARS-CoV genome can comprise a nucleotide sequence of at least 10 nucleotides located within a conserved region of SARS-CoV genome and a nucleotide sequence of at least 10 nucleotides located within a variable region of SARS-CoV genome. In another example, the at least two different nucleotide sequences of SARS-CoV genome can comprise a nucleotide sequence of at least 10 nucleotides located within a structural protein coding gene of SARS-CoV genome and a nucleotide sequence of at least 10 nucleotides located within a non-structural protein coding gene of SARS-CoV genome.

If desired, the present chips can comprise other types of probes or other features. For example, the chip can further comprise: a) at least one of the following three oligonucleotide probes: an immobilization control probe that is labeled and does not participate in any hybridization reaction when a sample containing or suspected of containing of a SARS-CoV is contacted with the chip, a positive control probe that is not complementary to any SARS-CoV sequence but is complementary to a non-SARS-CoV-sequence contained in the sample and a negative control probe that is not complementary to any nucleotide sequence contained in the sample; and b) a blank spot.

In a specific embodiment, the present chips can comprise at least two oligonucleotide probes complementary to two different nucleotide sequences of at least 10 nucleotides, respectively, located within a conserved region of SARS-CoV genome, located within a structural protein coding gene of SARS-CoV genome or located within a non-structural protein coding gene of SARS-CoV genome.

Any conserved region of SARS-CoV genome can be used as assay target. For example, the conserved region of SARS-CoV genome can be a region located within the Replicase 1A, 1B gene or the Nucleocapsid (N) gene of SARS-CoV.

Any variable region of SARS-CoV genome can be used as assay target. For example, the variable region of SARS-CoV genome can be a region located within the Spike glycoprotein (S) gene of SARS-CoV.

Any structural protein coding gene of SARS-CoV genome can be used as assay target. For example, the structural protein coding gene of SARS-CoV genome can be a gene encoding the Spike glycoprotein (S), the small envelope protein (E) or the Nucleocapsid protein (N).

Any non-structural protein coding gene of SARS-CoV genome can be used as assay target. For example, the non-structural protein coding gene of SARS-CoV genome can be a gene encoding the Replicase 1A or 1B.

In another specific embodiment, the present chips can comprise at least two of the following four oligonucleotide probes: two oligonucleotide probes complementary to two different nucleotide sequences of at least 10 nucleotides located within the Replicase 1A or 1B gene of SARS-CoV, an oligonucleotide probe complementary to a nucleotide sequence of at least 10 nucleotides located within the N gene of SARS-CoV and an oligonucleotide probe complementary to a nucleotide sequence of at least 10 nucleotides located within the S gene of SARS-CoV.

Preferably, one or both of the different nucleotide sequences located within the Replicase 1A or 1B gene of SARS-CoV can comprise a nucleotide sequence that: a) hybridizes, under high stringency, with a Replicase 1A or 1B nucleotide sequence, or a complementary strand thereof, that is set forth in Table 3; or b) has at least 90% identity to a Replicase 1A or 1B nucleotide sequence comprising a nucleotide sequence, or a complementary strand thereof, that is set forth in Table 3. More preferably, one or both of the different nucleotide sequences located within the Replicase 1A or 1B gene of SARS-CoV comprises a nucleotide sequence that is set forth in Table 3.

TABLE 3
Exemplary SARS-CoV probes

| | | |
|---|---|---|
| probe_id | Sequence 5′-3′ | region |

| | | |
|---|---|---|
| PBS00001 | TTACCCTAATATGTTTATCACCCGCGAAGAAGCTATTCGTCACGTTCGTGCGTGGA | SARS-Cov Replicase 1B |
| PBS00002 | CTGACAAGTATGTCCGCAATCTACAACACAGGCTCTATGAGTGTCTCTATAGAAAT | SARS-Cov Replicase 1B |
| PBS00003 | CATAACACTTGCTGTAACTTATCACACCGTTTCTACAGGTTAGCTAACGAGTGTGC | SARS-Cov Replicase 1B |
| PBS00004 | TTACCCTAATATGTTTATCACCCGCGAAGAAGCTATTCGTCACGTTCGT | SARS-Cov Replicase 1B |
| PBS00009 | GCGTTCTCTTAAAGCTCCTGCCGTAGTGTCAGTATCATCACCAGATGCTGTTACT | SARS-Cov Replicase 1A |
| | ACATATAATGGATAC | |
| PBS00010 | CTTTGGCTGGCTCTTACAGAGATTGGTCCTATTCAGGACAGCGTACAGAGTTAGG | SARS-Cov Replicase 1A |
| | TGTTGAATTTCTTAA | |
| PBS00011 | CTACGTAGTGAAGCTTTCGAGTACTACCATACTCTTGATGAGAGTTTTCTTGGTA | SARS-Cov Replicase 1A |
| | GGTACATGTCTGCTT | |
| PBS00012 | TGCCAATTGGTTATGTGACACATGGTTTTAATCTTGAAGAGGCTGCGCGCTGTAT | SARS-Cov Replicase 1A |
| | GCGTTCTCTTAAAGC | |
| PBS00013 | TATAAAGTTACCAAGGGAAAGCCCGTAAAAGGTGCTTGGAACATTGGACAACAGA | SARS-Cov Replicase 1A |
| | GATCAGTTTTAACAC | |
| PBS00014 | TGCTTCATTGATGTTGTTAACAAGGCACTCGAAATGTGCATTGATCAAGTCACTA | SARS-Cov Replicase 1A |
| | TCGCTGGCGCAAAG | |
| PBS00015 | TGTCGACGCCATGGTTTATACTTCAGACCTGCTCACCAACAGTGTCATTATTATG | SARS-Cov Replicase 1A |
| | GCATATGTAACTGGT | |
| PBS00016 | TACTGTTGAAAAACTCAGGCCTATCTTTGAATGGATTGAGGCGAAACTTAGTGCA | SARS-Cov Replicase 1A |
| | GGAGTTGAATTTCTC | |
| PBS00017 | ACCTATTCTGTTGCTTGACCAAGCTCTTGTATCAGACGTTGGAGATAGTACTGAA | SARS-Cov Replicase 1A |
| | GTTTCC | |
| PBS00018 | GCCTATTAATGTCATAGTTTTTGATGGCAAGTCCAAATGCGACGAGTCTGCTTCT | SARS-Cov Replicase 1A |
| | AAGTCTGCTTCTGTG | |
| PBS00019 | TGAGAGCTAACAACACTAAAGGTTCACTGCCTATTAATGTCATAGTTTTTGATGG | SARS-Cov Replicase 1A |
| | CAAGTCCAAATGCGA | |
| PBS00020 | ACTTGCATGATGTGCTATAAGCGCAATCGTGCCACACGCGTTGAGTGTACAACTA | SARS-Cov Replicase 1A |
| | TTGTTAATGGCATGA | |
| PBS00021 | GGCGATGTAGTGGCTATTGACTATAGACACTATTCAGCGAGTTTCAAGAAAGGTG | SARS-Cov Replicase 1A |
| | CTAAATTACTGCATA | |
| PBS00022 | TCAAACCAAACACTTGGTGTTTACGTTGTCTTTGGAGTACAAAGCCAGTAGATAC | SARS-Cov Replicase 1A |
| | TTCAAATTCATTTGA | |
| PBS00023 | TAGTGCTGTTGGCAACATTTGCTACACACCTTCCAAACTCATTGAGTATAGTGAT | SARS-Cov Replicase 1A |
| | TTTGCTAC | |
| PBS00024 | TCATAGCTAACATCTTTACTCCTCTTGTGCAACCTGTGGGTGCTTTAGATGTGTC | SARS-Cov Replicase 1A |
| | TGCTTCAGTAGTGGC | |
| PBS00025 | GGTATTATTGCCATATTGGTGACTTGTGCTGCCTACTACTTTATGAAATTCAGAC | SARS-Cov Replicase 1A |
| | GTGTTTTTGGTGAGT | |
| PBS00026 | GTGATGTCAGAGAAACTATGACCCATCTTCTACAGCATGCTAATTTGGAATCTGC | SARS-Cov Replicase 1A |
| | AAAGCGAGTTCTTAA | |
| PBS00027 | AACCATCAAGCCTGTGTCGTATAAACTCGATGGAGTTACTTACACAGAGATTGAA | SARS-Cov Replicase 1A |
| | CCAAAATTGGATGGG | |
| PBS00028 | GTTTTCTACAAGGAAACATCTTACACTACAACCATCAAGCCTGTGTCGTATAAAC | SARS-Cov Replicase 1A |
| | TCGATGGAGTTACTT | |
| PBS00029 | CCTTGAATGAGGATCTCCTTGAGATACTGAGTCGTGAACGTGTTAACATTAACAT | SARS-Cov Replicase 1A |
| | TGTTGGCGATTTTCA | |
| PBS00031 | GCCATGGTTTATACTTCAGACCTGCTCACCAACAGTGTCATTATTATGGCATATG | SARS-Cov Replicase 1A |
| | TAACTGGTGGTCTTG | |
| PBS00032 | CAACAGACTTCTCAGTGGTTGTCTAATCTTTTGGGCACTACTGTTGAAAAACTCA | SARS-Cov Replicase 1A |
| | GGCCTATCTTTGAAT | |
| PBS00033 | TTCCCGTCAGGCAAAGTTGAAGGGTGCATGGTACAAGTAACCTGTGGAACTACAAC | SARS-Cov Replicase 1A |
| PBS00034 | GGTTCACCATCTGGTGTTTATCAGTGTGCCATGAGACCTAATCATACCATTAAAGG | SARS-Cov Replicase 1A |
| PBS00035 | AGATCATGTTGACATATTGGGACCTCTTTCTGCTCAAACAGGAATTGCCGTC | SARS-Cov Replicase 1A |
| PBS00036 | TAAAAAGGACAAAAAGAAAAAGACTGATGAAGCTCAGCCTTTGCCGCAGAGACAA | SARS-Cov Nucleocapsid gene |
| PBS00037 | ACGGCAAAATGAAAGAGCTCAGCCCCAGATGGTACTTCTATTACCTAGGAACTGG | SARS-Cov Nucleocapsid gene |
| | CCCAGAAGCTTCACT | |
| PBS00038 | GGCGCTAACAAAGAAGGCATCGTATGGGTTGCAACTGAGGGAGCCTTGAATACAC | SARS-Cov Nucleocapsid gene |
| | CCAAAGACCACATTG | |
| PBS00039 | GTCCAGATGACCAAATTGGCTACTACCGAAGAGCTACCCGACGAGTTCGTGGTGG | SARS-Cov Nucleocapsid gene |
| | TGACGGCAAAATGAA | |
| PBS00040 | GAGGTGGTGAAACTGCCCTCGCGCTATTGCTGCTAGACAGATTGAACCAGCTTGA | SARS-Cov Nucleocapsid gene |
| | GAGCAAAGTTTCTGG | |
| PBS00041 | AAAAGAAAAAGACTGATGAAGCTCAGCCTTTGCCGCAGAGACAAAAGAAGCAGCC | SARS-Cov Nucleocapsid gene |
| | CACTGTGACTCTTCT | |
| PBS00042 | AAATTGCACAATTTGCTCCAAGTGCCTCTGCATTCTTTGGAATGTCACGCATTGG | SARS-Cov Nucleocapsid gene |
| | CATGGAAGTCACACC | |
| PBS00043 | ACCAATTTAACAAGGCGATTAGTCAAATTCAAGAATCACTTACAACAACATCAAC | SARS-Cov Spike glycoprotein gene |
| | TGCATTGGGCAAGCT | |
| PBS00044 | CACCTGGAACAAATGCTTCATCTGAAGTTGCTGTTCTATATCAAGATGTTAACTG | SARS-Cov Spike glycoprotein gene |
| | CACTGATGTTTCTAC | |
| PBS00045 | AAAGGGCTACCACCTTATGTCCTTCCCACAAGCAGCCCCGCATGGTGTTGTCTTC | SARS-Cov Spike glycoprotein gene |
| | CTACATGTCACGTAT | |
| PBS00046 | TCAGGAAATTGTGATGTCGTTATTGGCATCATTAACAACACAGTTTATGATCCTC | SARS-Cov Spike glycoprotein gene |
| | TGCAACCTGAGCTTG | |
| PBS00047 | TTGATCTTGGCGACATTTCAGGCATTAACGCTTCTGTCGTCAACATTCAAAAAGA | SARS-Cov Spike glycoprotein gene |
| | AATTGACCGCCTCAA | |
| PBS00048 | GAGGAACTTCACCACAGCGCCAGCAATTTGTCATGAAGGCAAAGCATACTTCCCT | SARS-Cov Spike glycoprotein gene |
| | CGTGAAGGTGTTTTT | |

Also preferably, the nucleotide sequence located within the N gene of SARS-CoV can comprise a nucleotide sequence that: a) hybridizes, under high stringency, with a N nucleotide sequence, or a complementary strand thereof, that is set forth in Table 3; or b) has at least 90% identity to a N nucleotide sequence comprising a nucleotide sequence, or a complementary strand thereof, that is set forth in Table 3. More preferably, the nucleotide sequence located within the N gene of SARS-CoV comprises a nucleotide sequence that is set forth in Table 3.

Also preferably, the nucleotide sequence located within the S gene of SARS-CoV can comprise a nucleotide sequence that: a) hybridizes, under high stringency, with a S nucleotide sequence, or a complementary strand thereof, that is set forth in Table 3; or b) has at least 90% identity to a S nucleotide sequence comprising a nucleotide sequence, or a complementary strand thereof, that is set forth in Table 3. More preferably, the nucleotide sequence located within the S gene of SARS-CoV comprises a nucleotide sequence that is set forth in Table 3.

Any suitable label can be used in the immobilization control probe, e.g., a chemical, an enzymatic, an immunogenic, a radioactive, a fluorescent, a lurminescent or a FRET label.

Any suitable non-SARS-CoV-sequence can be used. For example, the non-SARS-CoV-sequence can be an endogenous component of a sample to be assayed. Alternatively, the non-SARS-CoV-sequence is spiked in the sample to be assayed. In another example, the spiked non-SARS-CoV-sequence can be a sequence of Arabidopsis origin.

In still another specific embodiment, the present chips can comprise two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe that is labeled and does not participate in any hybridization reaction when a sample containing or suspected of containing of a SARS-CoV is contacted with the chip, a positive control probe that is not complementary to any SARS-CoV sequence but is complementary to a non-SARS-CoV-sequence contained in the sample and a negative control probe that is not complementary to any nucleotide sequence contained in the sample.

Preferably, the chip comprises multiple spots of the described probes, e.g. multiple spots of the two oligonucleotide probes complementary to two different nucleotide sequences located within the Replicase 1A or 1B gene of SARS-CoV, the oligonucleotide probe complementary to a nucleotide sequence located within the N gene of SARS-CoV, the oligonucleotide probe complementary to a nucleotide sequence located within the S gene of SARS-CoV, the immobilization control probe, the positive control probe and the negative control probe.

The present chips can further comprise an oligonucleotide probe complementary to a nucleotide sequence of a coronaviruse not related to the SARS-CoV. For example, the coronaviruse not related to the SARS can be the Group I, II or III coronaviruse or is a coronaviruse that infects an avian species, e.g., Avian infectious bronchitis virus and Avian infectious laryngotracheitis virus, an equine species, e.g., Equine coronaviruse, a canine species, e.g., Canine coronaviruse, a feline species, e.g., Feline coronaviruse and Feline infectious peritonitis virus, a porcine species, e.g., Porcine epidemic diarrhea virus, Porcine transmissible gastroenteritis virus and Porcine hemagglutinating encephalomyelitis virus, a calf species, e.g., Neonatal calf diarrhea coronaviruse, a bovine species, e.g., Bovine coronaviruse, a murine species, e.g., Murine hepatitis virus, a puffinosis species, e.g., Puffinosis virus, a rat species, e.g., Rat coronaviruse and a Sialodacryoadenitis virus of rat, e.g., a turkey species e.g., Turkey coronaviruse, or a human species, e.g., Human enteric coronaviruse. The present chips can further comprise an oligonucleotide probe complementary to a nucleotide sequence of other types of virus or pathogens. An exemplary list of viruses and pathogens that can be assayed using the present chips is set forth in the following Table 5.

TABLE 5
Exemplary viruses and pathogens

| | | | Sample | |
|---|---|---|---|---|
| No. | Virus name | Genome | nucleic acid | Structure |
| 1 | Coronaviridae | Single- | RNA | Having |
| | | stranded, | | capsid |
| | | linear RNA | | |
| 2 | SARS-CoV | Single- | RNA | Having |
| | | stranded, | | capsid |
| | | linear RNA | | |
| 3 | Human coronaviruse | Single- | RNA | Having |
| | 229E | stranded, | | capsid |
| | | linear RNA | | |
| 4 | Human coronaviruse | Single- | RNA | Having |
| | OC43 | stranded, | | capsid |
| | | linear RNA | | |
| 5 | Influenzavirus A, B, | Single- | RNA | Having |
| | C | stranded, | | capsid |
| | | linear RNA, | | |
| | | fragmented | | |
| 6 | Parainfluenza virus | Single- | RNA | Having |
| | | stranded, | | capsid |
| | | linear RNA | | |
| 7 | Respiratory sncytical | Single- | RNA | Having |
| | virus | stranded, | | capsid |
| | | linear RNA | | |
| 8 | Human | Single- | RNA | Having |
| | metapneumovirus | stranded, | | capsid |
| | | linear RNA | | |
| 9 | Rhinovirus | Single- | RNA | No capsid |
| | | stranded | | |
| | | RNA | | |
| 10 | Adenoviruse | Double- | DNA | No capsid |
| | | stranded, | | |
| | | linear DNA | | |
| 11 | Mycoplasma | Double- | DNA and | Having |
| | pneumoniae | stranded, | RNA | cell |
| | | linear DNA | | wall |
| 12 | Chlamydia pneumoniae | Double- | DNA and | No cell |
| | | stranded, | RNA | wall |
| | | linear DNA | | |

The various probes, e.g., the oligonucleotide probe complementary to a nucleotide sequence located within a conserved region of SARS-CoV genome, the oligonucleotide probe complementary to a nucleotide sequence located within a variable region of SARS-CoV genome, the immobilization control probe, the positive control probe or the negative control probe can comprise, at its ′5 end, a poly dT region to enhance its immobilization on the support.

In a specific embodiment, the at least one of the oligonucleotide probes is complementary to a highly expressed nucleotide sequence of SARS-CoV genome. Such a chip is particularly useful in detecting early-stage SARS-CoV infection.

The oligonucleotide probes and the target SARS-CoV nucleotide sequences can be any suitable length. Preferably, the oligonucleotide probes and the target SARS-CoV nucleotide sequences have a length of at least 7, 10, 20, 30, 40, 50, 60, 80, 90, 100 or more than 100 nucleotides.

The oligonucleotide probes and primers can be prepared by any suitable methods, e.g., chemical synthesis, recombinant methods and/or both (See generally, Ausubel et al., (Ed.), Current Protocols in Molecular Biology, John Wiley & Sons, Inc. (2000)).

Any suitable support can be used in the present chips. For example, the support can comprise a surface that is selected from the group consisting of a silicon, a plastic, a glass, a ceramic, a rubber, and a polymer surface.

### C. METHODS FOR ASSAYING FOR A SARS-COV IN A SAMPLE

In another aspect, the present invention is directed to a method for assaying for a SARS-CoV in a sample, which methods comprises: a) providing an above-described chip; b) contacting said chip with a sample containing or suspected of containing a SARS-CoV nucleotide sequence under conditions suitable for nucleic acid hybridization; and c) assessing hybrids formed between said SARS-CoV nucleotide sequence, if present in said sample, and said at least two oligonucleotide probes complementary to two different nucleotide sequences of SARS-CoV genome, respectively, to determine the presence, absence or amount of said SARS-CoV in said sample, whereby detection of one or both said hybrids indicates the presence of said SARS-CoV in said sample.

In a specific embodiment, the present methods comprise: a) providing a chip comprising a nucleotide sequence of at least 10 nucleotides located within a conserved region of SARS-CoV genome and a nucleotide sequence of at least 10 nucleotides located within a variable region of SARS-CoV genome, or a nucleotide sequence of at least 10 nucleotides located within a structural protein coding gene of SARS-CoV genome and a nucleotide sequence of at least 10 nucleotides located within a non-structural protein coding gene of SARS-CoV genome; b) contacting said chip with a sample containing or suspected of containing a SARS-CoV nucleotide sequence under conditions suitable for nucleic acid hybridization; and c) assessing hybrids formed between said SARS-CoV nucleotide sequence, if present in said sample, and i) said oligonucleotide probe complementary to a nucleotide sequence located within a conserved region of SARS-CoV genome and an oligonucleotide probe complementary to a nucleotide sequence located within a variable region of SARS-CoV genome, respectively; or ii) said oligonucleotide probe complementary to a nucleotide sequence located within a structural protein coding gene of SARS-CoV genome and an oligonucleotide probe complementary to a nucleotide sequence located within a non-structural protein coding gene of SARS-CoV genome, to determine the presence, absence or amount of said SARS-CoV in said sample, whereby detection of one or both said hybrids indicates the presence of said SARS-CoV in said sample.

In another specific embodiment, the present methods comprise: a) providing a chip comprising an oligonucleotide probe complementary to a nucleotide sequence within a conserved region of SARS-COV genome, an oligonucleotide probe, complementary to a nucleotide sequence located within a variable region of SARS-CoV genome, at least one of the following three oligonucleotide probes: an immobilization control probe that is labeled and does not participate in any hybridization reaction when a sample containing or suspected of containing of a SARS-CoV is contacted with the chip, a positive control probe that is not complementary to any SARS-CoV sequence but is complementary to a non-SARS-CoV-sequence contained in the sample and a negative control probe that is not complementary to any nucleotide sequence contained in the sample, and a blank spot; b) contacting said chip with a sample containing or suspected of containing a SARS-CoV nucleotide sequence under conditions suitable for nucleic acid hybridization; and c) assessing: (i) hybrids formed between said SARS-CoV nucleotide sequence, if present in the sample, and the oligonucleotide probe complementary to a nucleotide sequence within a conserved region of SARS-CoV genome and an oligonucleotide probe complementary to a nucleotide sequence located within a variable region of SARS-CoV genome, respectively; (ii) a label comprised in the immobilization control probe, or a hybrid(s) involving the positive control probe and/or the negative control probe; and (iii) a signal at said blank spot to determine the presence, absence or amount of said SARS-CoV in a sample.

Preferably, the present chips comprise two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe, a positive control probe and a negative control probe and the presence of the SARS-CoV is determined when: a) a positive hybridization signal is detected using at least one of the two different nucleotide sequences located within the Replicase 1A or 1B gene of SARS-CoV, the oligonucleotide probe complementary to a nucleotide sequence located within the N gene of SARS-CoV and the oligonucleotide probe complementary to a nucleotide sequence located within the S gene of SARS-CoV; b) a positive signal is detected from the immobilization control probe; c) a positive hybridization signal is detected using the positive control probe; d) a positive hybridization signal is not detected using the negative control probe; and e) a positive hybridization signal is not detected at the blank spot.

The inclusion of a target sequence in a variable region of SARS-CoV enables an assessment of possible mutation of the SARS-CoV. For example, detecting a positive hybridization signal using at least one of the two different nucleotide sequences located within the Replicase 1A or 1B gene of SARS-CoV, or the oligonucleotide probe complementary to a nucleotide sequence located within the N gene of SARS-CoV, while not detecting a positive hybridization signal using the oligonucleotide probe complementary to a nucleotide sequence located within the S gene of SARS-CoV indicates a mutation(s) of the SARS-CoV.

The present methods can be used for any suitable prognosis and diagnosis purpose. In one example, the present method is used to positively identify SARS-CoV infected patients from a population of patients who have SARS-like symptoms, e.g., fever or elevated temperature, nonproductive cough, myalgia, dyspnea, elevated lactate dehydrogenase, hypocalcemia, and lymphopenia (Booth et al., JAMA, 2003 May 6; [epub ahead of print]). The present chips, methods and kits can further comprise assaying for elevated lactate dehydrogenase, hypocalcemia, and lymphopenia, etc.

In another example, a chip further comprising an oligonucleotide probe complementary to a nucleotide sequence of a coronaviruse not related to the SARS-CoV is used and the method is used to positively identify SARS-CoV infected patients from patients who have been infected with a coronaviruse not related to the SARS, e.g., a coronaviruse that infects an avian species, e.g., Avian infectious bronchitis virus and Avian infectious laryngotracheitis virus, an equine species, e.g., Equine coronaviruse, a canine species, e.g., Canine coronaviruse, a feline species, e.g., Feline coronaviruse and Feline infectious peritonitis virus, a porcine species, e.g., Porcine epidemic diarrhea virus, Porcine transmissible gastroenteritis virus and Porcine hemagglutinating encephalomyelitis virus, a calf species, e.g., Neonatal calf diarrhea coronaviruse, a bovine species, e.g., Bovine coronaviruse, a murine species, e.g., Murine hepatitis virus, a puffinosis species, e.g., Puffinosis virus, a rat species, e.g., Rat coronaviruse and a Sialodacryoadenitis virus of rat, e.g., a turkey species e.g., Turkey coronaviruse, or a human species, e.g., Human enteric coronaviruse.

In still another example, a chip comprising an oligonucleotide probes complementary to a highly expressed nucleotide sequence of SARS-CoV genome is used and the method is used to diagnose early-stage SARS patients, e.g., SARS patients who have been infected with SARS-CoV from about less than one day to about three days.

In yet another example, the present methods are used to monitor treatment of SARS, e.g., treatment with an interferon or an agent that inhibits the replication of a variety of RNA viruses such as ribavirin. The present methods can also be used to assess potential anti-SARS-CoV agent in a drug screening assay.

Any suitable SARS-CoV nucleotide sequence can be assayed. For example, the SARS-CoV nucleotide sequence to be assayed can be a SARS-CoV RNA genomic sequence or a DNA sequence amplified from an extracted SARS-CoV RNA genomic sequence.

The SARS-CoV RNA genomic sequence can be prepared by any suitable methods. For example, the SARS-CoV RNA genomic sequence can be extracted from a SARS-CoV infected cell or other materials using the QIAamp Viral RNA kit, the Chomczynski-Sacchi technique or TRIzol (De Paula et al., J. Virol. Methods, 98(2):119-25 (2001)). Preferably, the SARS-COV RNA genomic sequence is extracted from a SARS-CoV infected cell or other materials using the QIAamp Viral RNA kit. The SARS-CoV RNA genomic sequence can be extracted from any suitable source. For example, the SARS-CoV RNA genomic sequence can be extracted from a sputum or saliva sample. In another example, the SARS-CoV RNA genomic sequence can be extracted from a lymphocyte of a blood sample.

The SARS-CoV RNA genomic sequence can be amplified by any suitable methods, e.g., PCR. Preferably, a label is incorporated into the amplified DNA sequence during the PCR. Any suitable PCR can be used, e.g., conventional, multiplex, nested PCR or RT-PCR. In one example, the PCR can comprise a two-step nested PCR, the first step being a RT-PCR and the second step being a conventional PCR. In another example, the PCR can comprise a one-step, multiplex RT-PCR using a plurality of 5′ and 3′ specific primers, each of the specific primers comprising a specific sequence complementary to its target sequence to be amplified and a common sequence, and a 5′ and a 3′ universal primer, the 5′ universal primer being complementary to the common sequence of the 5′ specific primers and the 3′ universal primer being complementary to the common sequence of the 3′ specific primers, and wherein in the PCR, the concentration of the 5′ and 3′ universal primers equals to or is higher than the concentration of the 5′ and 3′ specific primers, respectively. Preferably, the 3′ universal primer and/or the 5′ universal primer is labeled, e.g., a fluorescent label. In still another example, the PCR comprises a multiple step nested PCR or RT-PCR. In yet another example, the PCR is conducted using at least one of the following pairs of primers set forth in Table 4.

TABLE 4
Exemplary SARS-CoV primers

| | | |
|---|---|---|
| id | sequence (5′-3′) | region |

| | | |
|---|---|---|
| PMSL_00005 | CACGTCTCCCAAATGCTTGAGTGACG | SARS-Cov Nucleocapsid gene |
| PMSU_00006 | CCTCGAGGCCAGGGCGTTCC | SARS-Cov Nucleocapsid gene |
| PMV_00039 | TCACTTGCTTCCGTTGAGGTCGGGGACCAAGACCTAATCAGA | SARS-Cov Nucleocapsid gene |
| PMV_00040 | GGTTTCGGATGTTACAGCGTAGCCGCAGGAAGAAGAGTCACAG | SARS-Cov Nucleocapsid gene |
| PMV_00041 | TCACTTGCTTCCGTTGAGGAGGCCAGGGCGTTCCAATC | SARS-Cov Nucleocapsid gene |
| PMV_00042 | GGTTTCGGATGTTACAGCGTCAATAGCGCGAGGGCAGTTTC | SARS-Cov Nucleocapsid gene |
| PMV_00043 | TCACTTGCTTCCGTTGAGGGGCACCCGCAATCCTAATAACAA | SARS-Cov Nucleocapsid gene |
| PMV_00044 | GGTTTCGGATGTTACAGCGTAGCCGCAGGAAGAAGAGTCACAG | SARS-Cov Nucleocapsid gene |
| PMV_00090 | TCGGGGACCAAGACCTAATCAGA | SARS-Cov Nucleocapsid gene |
| PMV_00091 | AGCCGCAGGAAGAAGAGTCACAG | SARS-Cov Nucleocapsid gene |
| PMV_00092 | AGGCCAGGGCGTTCCAATC | SARS-Cov Nucleocapsid gene |
| PMV_00093 | CAATAGCGCGAGGGCAGTTTC | SARS-Cov Nucleocapsid gene |
| PMV_00094 | GGCACCCGCAATCCTAATAACAA | SARS-Cov Nucleocapsid gene |
| PMV_00095 | AGCCGCAGGAAGAAGAGTCACAG | SARS-Cov Nucleocapsid gene |
| PMSL_00001 | ACATCACAGCTTCTACACCCGTTAAGGT | SARS-Cov Replicase 1A |
| PMSL_00002 | ATACAGAATACATAGATTGCTGTTATCC | SARS-Cov Replicase 1A |
| PMSU_00002 | GCATCGTTGACTATGGTGTCCGATTCT | SARS-Cov Replicase 1A |
| PMSU_00003 | GCTGCATTGGTTTGTTATATCGTTATGC | SARS-Cov Replicase 1A |
| PMV_00023 | TCACTTGCTTCCGTTGAGGAGCCGCTTGTCACAATGCCAATT | SARS-Cov Replicase 1A |
| PMV_00024 | GGTTTCGGATGTTACAGCGTCATCACCAAGCTCGCCAACAGTT | SARS-Cov Replicase 1A |
| PMV_00025 | TCACTTGCTTCCGTTGAGGAGGTTGCCATCATTTTGGCATCTT | SARS-Cov Replicase 1A |
| PMV_00026 | GGTTTCGGATGTTACAGCGTCTTTGCGCCAGCGATAGTGACTT | SARS-Cov Replicase 1A |
| PMV_00027 | TCACTTGCTTCCGTTGAGGATGGCACCCGTTTCTGCAATGG | SARS-Cov Replicase 1A |
| PMV_00028 | GGTTTCGGATGTTACAGCGTTCGGGCAGCTGACACGAATGTAGA | SARS-Cov Replicase 1A |
| PMV_00029 | TCACTTGCTTCCGTTGAGGGAATGGCGATGTAGTGGCTATTGA | SARS-Cov Replicase 1A |
| PMV_00030 | GGTTTCGGATGTTACAGCGTTAATGCCGGCATCCAAACATAAT | SARS-Cov Replicase 1A |
| PMV_00031 | TCACTTGCTTCCGTTGAGGTAGCCAGCGTGGTGGTTCATACAA | SARS-Cov Replicase 1A |
| PMV_00032 | GGTTTCGGATGTTACAGCGTCTCCCGGCAGAAAGCTGTAAGCT | SARS-Cov Replicase 1A |
| PMV_00033 | TCACTTGCTTCCGTTGAGGTATAGAGCCCGTGCTGGTGATGC | SARS-Cov Replicase 1A |
| PMV_00034 | GGTTTCGGATGTTACAGCGTATCGCCATTCAAGTCTGGGAAGAA | SARS-Cov Replicase 1A |
| PMV_00035 | TCACTTGCTTCCGTTGAGGTGGCTCAGGCCATACTGGCATTAC | SARS-Cov Replicase 1A |
| PMV_00036 | GGTTTCGGATGTTACAGCGTTTTGCGCCAGCGATAGTGACTTG | SARS-Cov Replicase 1A |
| PMV_00037 | TCACTTGCTTCCGTTGAGGTTCCCGTCAGGCAAAGTTGAAGG | SARS-Cov Replicase 1A |
| PMV_00038 | GGTTTCGGATGTTACAGCGTGACGGCAATTCCTGTTTGAGCAGA | SARS-Cov Replicase 1A |
| PMV_00074 | AGCCGCTTGTCACAATGCCAATT | SARS-Cov Replicase 1A |
| PMV_00075 | CATCACCAAGCTCGCCAACAGTT | SARS-Cov Replicase 1A |
| PMV_00076 | AGGTTGCCATCATTTTGGCATCTT | SARS-Cov Replicase 1A |
| PMV_00077 | CTTTGCGCCAGCGATAGTGACTT | SARS-Cov Replicase 1A |
| PMV_00078 | ATGGCACCCGTTTCTGCAATGG | SARS-Cov Replicase 1A |
| PMV_00079 | TCGGGCAGCTGACACGAATGTAGA | SARS-Cov Replicase 1A |
| PMV_00080 | GAATGGCGATGTAGTGGCTATTGA | SARS-Cov Replicase 1A |
| PMV_00081 | TAATGCCGGCATCCAAACATAAT | SARS-Cov Replicase 1A |
| PMV_00082 | TAGCCAGCGTGGTGGTTCATACAA | SARS-Cov Replicase 1A |
| PMV_00083 | CTCCCGGCAGAAAGCTGTAAGCT | SARS-Cov Replicase 1A |
| PMV_00084 | TATAGAGCCCGTGCTGGTGATGC | SARS-Cov Replicase 1A |
| PMV_00085 | ATCGCCATTCAAGTCTGGGAAGAA | SARS-Cov Replicase 1A |
| PMV_00086 | TGGCTCAGGCCATACTGGCATTAC | SARS-Cov Replicase 1A |
| PMV_00087 | TTTGCGCCAGCGATAGTGACTTG | SARS-Cov Replicase 1A |
| PMV_00088 | TTCCCGTCAGGCAAAGTTGAAGG | SARS-Cov Replicase 1A |
| PMV_00089 | GACGGCAATTCCTGTTTGAGCAGA | SARS-Cov Replicase 1A |
| PMV_00003 | TCACTTGCTTCCGTTGAGGATGAATTACCAAGTCAATGGTTAC | SARS-Cov Replicase 1B |
| PMV_00004 | GGTTTCGGATGTTACAGCGTATAACCAGTCGGTACAGCTAC | SARS-Cov Replicase 1B |
| PMV_00005 | TCACTTGCTTCCGTTGAGGGAAGCTATTCGTCACGTTCG | SARS-Cov Replicase 1B |
| PMV_00006 | GGTTTCGGATGTTACAGCGTCTGTAGAAAATCCTAGCTGGAG | SARS-Cov Replicase 1B |
| PMV_00007 | TCACTTGCTTCCGTTGAGGCCTCTCTTGTTCTTGCTCGCA | SARS-Cov Replicase 1B |
| PMV_00008 | GGTTTCGGATGTTACAGCGTGTGAGCCGCCACACATG | SARS-Cov Replicase 1B |
| PMV_00009 | TCACTTGCTTCCGTTGAGGCTAACATGCTTAGGATAATGG | SARS-Cov Replicase 1B |
| PMV_00010 | GGTTTCGGATGTTACAGCGTCAGGTAAGCGTAAAACTCATC | SARS-Cov Replicase 1B |
| PMV_00011 | TCACTTGCTTCCGTTGAGGGCCTCTCTTGTTCTTGCTCGC | SARS-Cov Replicase 1B |
| PMV_00013 | TCACTTGCTTCCGTTGAGGCACCGTTTCTACAGGTTAGCTAACGA | SARS-Cov Replicase 1B |
| PMV_00014 | GGTTTCGGATGTTACAGCGTAAATGTTTACGCAGGTAAGCGTAAAA | SARS-Cov Replicase 1B |
| PMV_00015 | TCACTTGCTTCCGTTGAGGTACACACCTCAGCGTTG | SARS-Cov Replicase 1B |
| PMV_00016 | GGTTTCGGATGTTACAGCGTCACGAACGTGACGAAT | SARS-Cov Replicase 1B |
| PMV_00017 | TCACTTGCTTCCGTTGAGGGCTTAGGATAATGGCCTCTC | SARS-Cov Replicase 1B |
| PMV_00018 | GGTTTCGGATGTTACAGCGTCCACGAATTCATGATCAACATCCC | SARS-Cov Replicase 1B |
| PMV_00019 | TCACTTGCTTCCGTTGAGGGCTCGCAAACATAACACTTGC | SARS-Cov Replicase 1B |
| PMV_00020 | GGTTTCGGATGTTACAGCGTGAGACACTCATAGAGCCTGTG | SARS-Cov Replicase 1B |
| PMV_00055 | ATGAATTACCAAGTCAATGGTTAC | SARS-Cov Replicase 1B |
| PMV_00056 | ATAACCAGTCGGTACAGCTAC | SARS-Cov Replicase 1B |
| PMV_00057 | GAAGCTATTCGTCACGTTCG | SARS-Cov Replicase 1B |
| PMV_00058 | CTGTAGAAAATCCTAGCTGGAG | SARS-Cov Replicase 1B |
| PMV_00059 | CCTCTCTTGTTCTTGCTCGCA | SARS-Cov Replicase 1B |
| PMV_00060 | GTGAGCCGCCACACATG | SARS-Cov Replicase 1B |
| PMV_00061 | CTAACATGCTTAGGATAATGG | SARS-Cov Replicase 1B |
| PMV_00062 | CAGGTAAGCGTAAAACTCATC | SARS-Cov Replicase 1B |
| PMV_00063 | GCCTCTCTTGTTCTTGCTCGC | SARS-Cov Replicase 1B |
| PMV_00064 | CACCGTTTCTACAGGTTAGCTAACGA | SARS-Cov Replicase 1B |
| PMV_00065 | AAATGTTTACGCAGGTAAGCGTAAAA | SARS-Cov Replicase 1B |
| PMV_00066 | TACACACCTCAGCGTTG | SARS-Cov Replicase 1B |
| PMV_00067 | CACGAACGTGACGAAT | SARS-Cov Replicase 1B |
| PMV_00068 | GCTTAGGATAATGGCCTCTC | SARS-Cov Replicase 1B |
| PMV_00069 | CCACGAATTCATGATCAACATCC | SARS-Cov Replicase 1B |
| PMV_00070 | GCTCGCAAACATAACACTTGC | SARS-Cov Replicase 1B |
| PMV_00071 | GAGACACTCATAGAGCCTGTG | SARS-Cov Replicase 1B |
| PMSL_00003 | CCAGCTCCAATAGGAATGTCGCACTC | SARS-Cov Spike glycoprotein gene |
| PMSL_00004 | TCCGCAGATGTACATATTACAATCTACG | SARS-Cov Spike glycoprotein gene |
| PMSU_00005 | TTAAATGCACCGGCCACGGTTTG | SARS-Cov Spike glycoprotein gene |
| PMV_000100 | ATAGCGCCAGGACAAACTGGTGTT | SARS-Cov Spike glycoprotein gene |
| PMV_000101 | TATATGCGCCAAGCTGGTGTGAGT | SARS-Cov Spike glycoprotein gene |
| PMV_000102 | CGAGGCGGAGGTACAAATTGACAG | SARS-Cov Spike glycoprotein gene |
| PMV_000103 | ATGAAGCCGAGCCAAACATACCAA | SARS-Cov Spike glycoprotein gene |
| PMV_00045 | TCACTTGCTTCCGTTGAGGATGCACCGGCCACGGTTTGTG | SARS-Cov Spike glycoprotein gene |
| PMV_00046 | GGTTTCGGATGTTACAGCGTATGCGCCAAGCTGGTGTGAGTTGA | SARS-Cov Spike glycoprotein gene |
| PMV_00047 | TCACTTGCTTCCGTTGAGGTGCTGGCGCTGCTCTTCAAATACC | SARS-Cov Spike glycoprotein gene |
| PMV_00048 | GGTTTCGGATGTTACAGCGTCGGGGCTGCTTGTGGGAAGG | SARS-Cov Spike glycoprotein gene |
| PMV_00049 | TCACTTGCTTCCGTTGAGGATAGCGCCAGGACAAACTGGTGTT | SARS-Cov Spike glycoprotein gene |
| PMV_00050 | GGTTTCGGATGTTACAGCGTTATATGCGCCAAGCTGGTGTGAGT | SARS-Cov Spike glycoprotein gene |
| PMV_00051 | TCACTTGCTTCCGTTGAGGCGAGGCGGAGGTACAAATTGACAG | SARS-Cov Spike glycoprotein gene |
| PMV_00052 | GGTTTCGGATGTTACAGCGTATGAAGCCGAGCCAAACATACCAA | SARS-Cov Spike glycoprotein gene |
| PMV_00096 | ATGCACCGGCCACGGTTTGTG | SARS-Cov Spike glycoprotein gene |
| PMV_00097 | ATGCGCCAAGCTGGTGTGAGTTGA | SARS-Cov Spike glycoprotein gene |
| PMV_00098 | TGCTGGCGCTGCTCTTCAAATACC | SARS-Cov Spike glycoprotein gene |
| PMV_00099 | CGGGGCTGCTTGTGGGAAGG | SARS-Cov Spike glycoprotein gene |

### D. SARS-COV PRIMERS, PROBES, KIT AND USES THEREOF

In still another aspect, the present invention is directed to an oligonucleotide primer for amplifying a SARS-CoV nucleotide sequence, which oligonucleotide primer comprises a nucleotide sequence that: a) hybridizes, under high stringency, with a target SARS-CoV nucleotide sequence, or a complementary strand thereof, that is set forth in Table 1; or b) has at least 90% identity to a target SARS-CoV nucleotide sequence comprising a nucleotide sequence, or a complementary strand thereof, that is set forth in Table 1.

The present primers can comprise any suitable types of nucleic acids, e.g., DNA, RNA, PNA or a derivative thereof. Preferably, the primers comprise a nucleotide sequence, or a complementary strand thereof, that is set forth in Table 1.

In a specific embodiment, the present invention is directed to a kit for amplifying a SARS-CoV nucleotide sequence, which kit comprises: a) an above-described primer; and b) a nucleic acid polymerase that can amplify a SARS-CoV nucleotide sequence using the probe. Preferably, the nucleic acid polymerase is a reverse transcriptase.

In yet another aspect, the present invention is directed to an oligonucleotide probe for hybridizing to a SARS-CoV nucleotide sequence, which oligonucleotide probe comprises a nucleotide sequence that: a) hybridizes, under high stringency, with a target SARS-CoV nucleotide sequence, or a complementary strand thereof, that is set forth in Table 2; or b) has at least 90% identity to a target SARS-CoV nucleotide sequence comprising a nucleotide sequence, or a complementary strand thereof, that is set forth in Table 2.

The present probes can comprise any suitable types of nucleic acids, e.g., DNA, RNA, PNA or a derivative thereof. Preferably, the probes comprise a nucleotide sequence, or a complementary strand thereof, that is set forth in Table 2. Also preferably, the probes are labeled, e.g., a chemical, an enzymatic, an immunogenic, a radioactive, a fluorescent, a luminescent and a FRET label.

In a specific embodiment, the present invention is directed to a kit for hybridization analysis of a SARS-CoV nucleotide sequence, which kit comprises: a) an above-described probe; and b) a means for assessing a hybrid formed between a SARS-CoV nucleotide sequence and said probe.

The oligonucleotide primers and probes can be produced by any suitable method. For example, the probes can be chemically synthesized (See generally, Ausubel (Ed.) Current Protocols in Molecular Biology, 2.11. Synthesis and purification of oligonucleotides, John Wiley & Sons, Inc. (2000)), isolated from a natural source, produced by recombinant methods or a combination thereof. Synthetic oligonucleotides can also be prepared by using the triester method of Matteucci et al., J. Am. Chem. Soc., 3:3185-3191(1981). Alternatively, automated synthesis may be preferred, for example, on a Applied Biosynthesis DNA synthesizer using cyanoethyl phosphoramidite chemistry. Preferably, the probes and the primers are chemically synthesized.

Suitable bases for preparing the oligonucleotide probes and primers of the present invention may be selected from naturally occurring nucleotide bases such as adenine, cytosine, guanine, uracil, and thymine. It may also be selected from nonmaturally occurring or “synthetic” nucleotide bases such as 8-oxo-guanine, 6-mercaptoguanine, 4-acetylcytidine, 5-(carboxyhydroxyethyl) uridine, 2′-O-methylcytidine, 5-carboxymethylamino-methyl-2-thioridine, 5-carboxymethylaminomethyl uridine, dihydrouridine, 2′-O-methylpseudouridine, beta-D-galactosylqueosine, 2′-Omethylguanosine, inosine, N⁶-isopentenyladenosine, 1-methyladenosine; 1-methylpseudouridine, 1-methylguanosine, 1-methylinosine, 2,2-dimethylguanosine, 2-methyladenosine, 2-methylguanosine, 3-methylcytidine, 5-methylcytidine, N⁶-methyladenosine, 7-methylguanosine, 5-methylaminomethyluridine, 5-methoxyaminomethyl-2-thiouridine, beta-D-mannosylqueosine, 5-methoxycarbonylmethyluridine, 5-methoxyuridine, 2-methylthio-N⁶-isopentenyladenosine, N-((9-.beta.-D-ribofuranosyl-2-methylthiopurine-6-yl)carbamoyl)threonine, N-((9-beta-D-ribofuranosylpurine-6-yl) N-methylcarbamoyl) threonine, uridine-5-oxyacetic acid methylester, uridine-5-oxyacetic acid, wybutoxosine, pseudouridine, queosine, 2-thiocytidine, 5-methyl-2-thiouridine, 2-thiouridine, 2-thiouridine, 5-methyluridine, N-((9-beta-D-ribofuranosylpurine-6-yl) carbamoyl) threonine, 2′-O-methyl-5-methyluridine, 2′-O-methyluridine, wybutosine, and 3-(3-amino-3-carboxypropyl) uridine.

Likewise, chemical analogs of oligonucleotides (e.g., oligonucleotides in which the phosphodiester bonds have been modified, e.g., to the methylphosphonate, the phosphotriester, the phosphorothioate, the phosphorodithioate, or the phosphoramidate) may also be employed. Protection from degradation can be achieved by use of a “3′-end cap” strategy by which nuclease-resistant linkages are substituted for phosphodiester linkages at the 3′ end of the oligonucleotide (Shaw et al., Nucleic Acids Res., 19:747 (1991)). Phosphoramidates, phosphorothioates, and methylphosphonate linkages all function adequately in this manner. More extensive modification of the phosphodiester backbone has been shown to impart stability and may allow for enhanced affinity and increased cellular permeation of oligonucleotides (Milligan et al., J. Med. Chem., 36:1923 (1993)). Many different chemical strategies have been employed to replace the entire phosphodiester backbone with novel linkages. Backbone analogues include phosphorothioate, phosphorodithioate, methylphosphonate, phosphoramidate, boranophosphate, phosphotriester, formacetal, 3′-thioformacetal, 5′-thioformacetal, 5′-thioether, carbonate, 5′-N-carbamate, sulfate, sulfonate, sulfamate, sulfonamide, sulfone, sulfite, sulfoxide, sulfide, hydroxylamine, methylene (methylimino) (MMI) or methyleneoxy (methylimino) (OMI) linkages. Phosphorothioate and methylphosphonate-modified oligonucleotides are particularly preferred due to their availability through automated oligonucleotide synthesis. The oligonucleotide may be a “peptide nucleic acid” such as described by (Milligan et al., J. Med. Chem., 36:1923 (1993)). The only requirement is that the oligonucleotide probe should possess a sequence at least a portion of which is capable of binding to a portion of the sequence of a target SARS-CoV sequence.

Hybridization probes or amplification primers can be of any suitable length. There is no lower or upper limits to the length of the probe or primer, as long as the probe hybridizes to the SARS-CoV target nucleic acids and functions effectively as a probe or primer (e.g., facilitates detection or amplification). The probes and primers of the present invention can be as short as 50, 40, 30, 20, 15, or 10 nucleotides, or shorter. Likewise, the probes or primers can be as long as 20, 40, 50, 60, 75, 100 or 200 nucleotides, or longer, e.g., to the full length of the SARS-CoV target sequence. Generally, the probes will have at least 14 nucleotides, preferably at least 18 nucleotides, and more preferably at least 20 to 30 nucleotides of either of the complementary target nucleic acid strands and does not contain any hairpin secondary structures. In specific embodiments, the probe can have a length of at least 30 nucleotides or at least 50 nucleotides. If there is to be complete complementarity, i.e., if the strand contains a sequence identical to that of the probe, the duplex will be relatively stable under even stringent conditions and the probes may be short, i.e., in the range of about 10-30 base pairs. If some degree of mismatch is expected in the probe, i.e., if it is suspected that the probe would hybridize to a variant region, or to a group of sequences such as all species within a specific genus, the probe may be of greater length (i.e., 15-40 bases) to balance the effect of the mismatch(es).

The probe need not span the entire SARS-CoV target gene. Any subset of the target region that has the potential to specifically identify SARS-CoV target or alelle can be used. Consequently, the nucleic acid probe may hybridize to as few as 8 nucleotides of the target region. Further, fragments of the probes may be used so long as they are sufficiently characteristic of the SARS-CoV target gene to be typed.

The probe or primer should be able to hybridize with a SARS-CoV target nucleotide sequence that is at least 8 nucleotides in length under low stringency. Preferably, the probe or primer hybridizes with a SARS-CoV target nucleotide sequence under middle or high stringency.

In still another aspect, the present invention is directed to an array of oligonucleotide probes immobilized on a support for typing a SARS-CoV target gene, which array comprises a support suitable for use in nucleic acid hybridization having immobilized thereon a plurality of oligonucleotide probes, at least one of said probes comprising a nucleotide sequence that: a) hybridizes, under high stringency, with a target SARS-CoV nucleotide sequence, or a complementary strand thereof, that is set forth in Table 3; or b) has at least 90% identity to a target SARS-CoV nucleotide sequence comprising a nucleotide sequence, or a complementary strand thereof, that is set forth in Table 3.

The plurality of probes can comprise DNA, RNA, PNA or a derivative thereof. At least one or some of the probes can comprise a nucleotide sequence, or a complementary strand thereof, that is set forth in Table 1. Preferably, probe arrays comprise all of the nucleotide sequences, or a complementary strand thereof, that are set forth in Table 3. At least one, some or all of the probes can be labeled. Exemplary labels inlcude a chemical, an enzymatic, an immunogenic, a radioactive, a fluorescent, a luminescent and a FRET label. Any suitable support, e.g., a silicon, a plastic, a glass, a ceramic, a rubber, and a polymer surface, can be used.

### E. ASSAY FORMATS

Immobilization of Probes

The present methods, probes and probe arrays can be used in solution. Preferably, it is conducted in chip format, e.g., by using the probe(s) immobilized on a solid support.

The probes can be immobilized on any suitable surface, preferably, a solid support, such as silicon, plastic, glass, ceramic, rubber, or polymer surface. The probe may also be immobilized in a 3-dimensional porous gel substrate, e.g., Packard HydroGel chip (Broude et al., Nucleic Acids Res., 29(19):E92 (2001)).

For an array-based assay, the probes are preferably immobilized to a solid support such as a “biochip”. The solid support may be biological, nonbiological, organic, inorganic, or a combination of any of these, existing as particles, strands, precipitates, gels, sheets, tubing, spheres, containers, capillaries, pads, slices, films, plates, slides, etc.

A microarray biochip containing a library of probes can be prepared by a number of well known approaches including, for example, light-directed methods, such as VLSIPS™ described in U.S. Pat. Nos. 5,143,854, 5,384,261 or 5,561,071; bead based methods such as described in U.S. Pat. No. 5,541,061; and pin based methods such as detailed in U.S. Pat. No. 5,288,514. U.S. Pat. No. 5,556,752, which details the preparation of a library of different double stranded probes as a microarray using the VLSIPS™, is also suitable for preparing a library of hairpin probes in a microarray.

Flow channel methods, such as described in U.S. Pat. Nos. 5,677,195 and 5,384,261, can be used to prepare a microarray biochip having a variety of different probes. In this case, certain activated regions of the substrate are mechanically separated from other regions when the probes are delivered through a flow channel to the support. A detailed description of the flow channel method can be found in U.S. Pat. No. 5,556,752, including the use of protective coating wetting facilitators to enhance the directed channeling of liquids though designated flow paths.

Spotting methods also can be used to prepare a microarray biochip with a variety of probes immobilized thereon. In this case, reactants are delivered by directly depositing relatively small quantities in selected regions of the support. In some steps, of course, the entire support surface can be sprayed or otherwise coated with a particular solution. In particular formats, a dispenser moves from region to region, depositing only as much probe or other reagent as necessary at each stop. Typical dispensers include micropipettes, nanopippettes, ink-jet type cartridges and pins to deliver the probe containing solution or other fluid to the support and, optionally, a robotic system to control the position of these delivery devices with respect to the support. In other formats, the dispenser includes a series of tubes or multiple well trays, a manifold, and an array of delivery devices so that various reagents can be delivered to the reaction regions simultaneously. Spotting methods are well known in the art and include, for example, those described in U.S. Pat. Nos. 5,288,514, 5,312,233 and 6,024,138. In some cases, a combination of flow channels and “spotting” on predefined regions of the support also can be used to prepare microarray biochips with immobilized probes.

A solid support for immobilizing probes is preferably flat, but may take on alternative surface configurations. For example, the solid support may contain raised or depressed regions on which probe synthesis takes place or where probes are attached. In some embodiments, the solid support can be chosen to provide appropriate light-absorbing characteristics. For example, the support may be a polymerized Langmuir Blodgett film, glass or functionalized glass, Si, Ge, GaAs, GaP, SiO₂, SiN4, modified silicon, or any one of a variety of gels or polymers such as (poly)tetrafluoroethylene, (poly)vinylidendifluoride, polystyrene, polycarbonate, or combinations thereof. Other suitable solid support materials will be readily apparent to those of skill in the art.

The surface of the solid support can contain reactive groups, which include carboxyl, amino, hydroxyl, thiol, or the like, suitable for conjugating to a reactive group associated with an oligonucleotide or a nucleic acid. Preferably, the surface is optically transparent and will have surface Si--OH functionalities, such as those found on silica surfaces.

The probes can be attached to the support by chemical or physical means such as through ionic, covalent or other forces well known in the art. Immobilization of nucleic acids and oligonucleotides can be achieved by any means well known in the art (see, e.g., Dattagupta et al., Analytical Biochemistry, 177:85-89(1989); Saiki et al., Proc. Natl. Acad Sci. USA, 86:6230-6234(1989); and Gravitt et al., J. Clin. Micro., 36:3020-3027(1998)).

The probes can be attached to a support by means of a spacer molecule, e.g., as described in U.S. Pat. No. 5,556,752 to Lockhart et al., to provide space between the double stranded portion of the probe as may be helpful in hybridization assays. A spacer molecule typically comprises between 6-50 atoms in length and includes a surface attaching portion that attaches to the support. Attachment to the support can be accomplished by carbon-carbon bonds using, for example, supports having (poly)trifluorochloroethylene surfaces, or preferably, by siloxane bonds (using, for example, glass or silicon oxide as the solid support). Siloxane bonding can be formed by reacting the support with trichlorosilyl or trialkoxysilyl groups of the spacer. Aminoalkylsilanes and hydroxyalkylsilanes, bis(2-hydroxyethyl)-aminopropyltriethoxysilane, 2-hydroxyethylaminopropyltriethoxysilane, aminopropyltriethoxysilane or hydroxypropyltriethoxysilane are useful are surface attaching groups.

The spacer can also include an extended portion or longer chain portion that is attached to the surface-attaching portion of the probe. For example, amines, hydroxyl, thiol, and carboxyl groups are suitable for attaching the extended portion of the spacer to the surface-attaching portion. The extended portion of the spacer can be any of a variety of molecules which are inert to any subsequent conditions for polymer synthesis. These longer chain portions will typically be aryl acetylene, ethylene glycol oligomers containing 2-14 monomer units, diamines, diacids, amino acids, peptides, or combinations thereof.

In some embodiments, the extended portion of the spacer is a polynucleotide or the entire spacer can be a polynucleotide. The extended portion of the spacer also can be constructed of polyethyleneglycols, polynucleotides, alkylene, polyalcohol, polyester, polyamine, polyphosphodiester and combinations thereof Additionally, for use in synthesis of probes, the spacer can have a protecting group attached to a functional group (e.g., hydroxyl, amino or carboxylic acid) on the distal or terminal end of the spacer (opposite the solid support). After deprotection and coupling, the distal end can be covalently bound to an oligomer or probe.

The present method can be used to analyze a single sample with a single probe at a time. Preferably, the method is conducted in high-throughput format. For example, a plurality of samples can be analyzed with a single probe simultaneously, or a single sample can be analyzed using a plurality of probes simultaneously. More preferably, a plurality of samples can be analyzed using a plurality of probes simultaneously.

Hybridization Conditions

Hybridization can be carried out under any suitable technique known in the art. It will be apparent to those skilled in the art that hybridization conditions can be altered to increase or decrease the degree of hybridization, the level of specificity of the hybridization, and the background level of non-specific binding (i.e., by altering hybridization or wash salt concentrations or temperatures). The hybridization between the probe and the target nucleotide sequence can be carried out under any suitable stringencies, including high, middle or low stringency. Typically, hybridizations will be performed under conditions of high stringency.

Hybridization between the probe and target nucleic acids can be homogenous, e.g., typical conditions used in molecular beacons (Tyagi S. et al., Nature Biotechnology, 14:303-308 (1996); and U.S. Pat. No. 6,150,097 ) and in hybridization protection assay (Gen-Probe, Inc) (U.S. Pat. No. 6,004,745), or heterogeneous (typical conditions used in different type of nitrocellulose based hybridization and those used in magnetic bead based hybridization).

The target polynucleotide sequence may be detected by hybridization with an oligonucleotide probe that forms a stable hybrid with that of the target sequence under high to low stringency hybridization and wash conditions. An advantage of detection by hybridization is that, depending on the probes used, additional specificity is possible. If it is expected that the probes will be completely complementary (i.e., about 99% or greater) to the target sequence, high stringency conditions will be used. If some mismatching is expected, for example, if variant strains are expected with the result that the probe will not be completely complementary, the stringency of hybridization may be lessened. However, conditions are selected to minimize or eliminate nonspecific hybridization.

Conditions those affect hybridization and those select against nonspecific hybridization are known in the art (Molecular Cloning A Laboratory Manual, second edition, J. Sambrook, E. Fritsch, T. Maniatis, Cold Spring Harbor Laboratory Press, 1989). Generally, lower salt concentration and higher temperature increase the stringency of hybridization. For example, in general, stringent hybridization conditions include incubation in solutions that contain approximately 0.1×SSC, 0.1% SDS, at about 65° C. incubation/wash temperature. Middle stringent conditions are incubation in solutions that contain approximately 1-2×SSC, 0.1% SDS and about 50° C.-65° C. incubation/wash temperature. The low stringency conditions are 2×SSC and about 30° C.-50° C.

An alternate method of hybridization and washing is first to carry out a low stringency hybridization (5×SSPE, 0.5% SDS) followed by a high stringency wash in the presence of 3M tetramethyl-ammonium chloride (TMAC). The effect of the TMAC is to equalize the relative binding of A-T and G-C base pairs so that the efficiency of hybridization at a given temperature corresponds more closely to the length of the polynucleotide. Using TMAC, it is possible to vary the temperature of the wash to achieve the level of stringency desired (Wood et al., Proc. Natl. Acad. Sci. USA, 82:1585-1588 (1985)).

A hybridization solution may contain 25% formamide, 5×SSC, 5× Denhardt's solution, 100 μg/ml of single stranded DNA, 5% dextran sulfate, or other reagents known to be useful for probe hybridization.

Detection of the Hybrid

Detection of hybridization between the probe and the target SARS-CoV nucleic acids can be carried out by any method known in the art, e.g., labeling the probe, the secondary probe, the target nucleic acids or some combination thereof, and are suitable for purposes of the present invention. Alternatively, the hybrid may be detected by mass spectroscopy in the absence of detectable label (e.g., U.S. Pat. No. 6,300,076).

The detectable label is a moiety that can be detected either directly or indirectly after the hybridization. In other words, a detectable label has a measurable physical property (e.g., fluorescence or absorbance) or is participant in an enzyme reaction. Using direct labeling, the target nucleotide sequence or the probe is labeled, and the formation of the hybrid is assessed by detecting the label in the hybrid. Using indirect labeling, a secondary probe is labeled, and the formation of the hybrid is assessed by the detection of a secondary hybrid formed between the secondary probe and the original hybrid.

Methods of labeling probes or nucleic acids are well known in the art. Suitable labels include fluorophores, chromophores, luminophores, radioactive isotopes, electron dense reagents, FRET(fluorescence resonance energy transfer), enzymes and ligands having specific binding partners. Particularly useful labels are enzymatically active groups such as enzymes (Wisdom, Clin. Chem., 22:1243 (1976)); enzyme substrates (British Pat. No. 1,548,741); coenzymes (U.S. Pat. Nos. 4,230,797 and 4,238,565) and enzyme inhibitors (U.S. Pat. No.4,134,792); fluorescers (Soini and Hemmila, Clin. Chem., 25:353 (1979)); chromophores including phycobiliproteins, luminescers such as chemiluminescers and bioluminescers (Gorus and Schram, Clin. Chem., 25:512 (1979) and ibid, 1531); specifically bindable ligands, i.e., protein binding ligands; antigens; and residues comprising radioisotopes such as ³H, ³⁵S, ³²P, ¹²⁵I, and ¹⁴C Such labels are detected on the basis of their own physical properties (e.g., fluorescers, chromophores and radioisotopes) or their reactive or binding properties (e.g., antibodies, enzymes, substrates, coenzymes and inhibitors). Ligand labels are also useful for solid phase capture of the oligonucleotide probe (i.e., capture probes). Exemplary labels include biotin (detectable by binding to labeled avidin or streptavidin) and enzymes, such as horseradish peroxidase or alkaline phosphatase (detectable by addition of enzyme substrates to produce a colored reaction product).

For example, a radioisotope-labeled probe or target nucleic acid can be detected by autoradiography. Alternatively, the probe or the target nucleic acid labeled with a fluorescent moiety can detected by fluorimetry, as is known in the art. A hapten or ligand (e.g., biotin) labeled nucleic acid can be detected by adding an antibody or an antibody pigment to the hapten or a protein that binds the labeled ligand (e.g., avidin).

As a further alternative, the probe or nucleic acid may be labeled with a moiety that requires additional reagents to detect the hybridization. If the label is an enzyme, the labeled nucleic acid, e.g., DNA, is ultimately placed in a suitable medium to determine the extent of catalysis. For example, a cofactor-labeled nucleic acid can be detected by adding the enzyme for which the label is a cofactor and a substrate for the enzyme. Thus, if the enzyme is a phosphatase, the medium can contain nitrophenyl phosphate and one can monitor the amount of nitrophenol generated by observing the color. If the enzyme is a beta-galactosidase, the medium can contain o-nitro-phenyl-D-galacto-pyranoside, which also liberates nitrophenol. Exemplary examples of the latter include, but are not limited to, beta-galactosidase, alkaline phosphatase, papain and peroxidase. For in situ hybridization studies, the final product of the substrate is preferably water insoluble. Other labels, e.g., dyes, will be evident to one having ordinary skill in the art.

The label can be linked directly to the DNA binding ligand, e.g., acridine dyes, phenanthridines, phenazines, furocoumarins, phenothiazines and quinolines, by direct chemical linkage such as involving covalent bonds, or by indirect linkage such as by the incorporation of the label in a microcapsule or liposome, which in turn is linked to the binding ligand. Methods by which the label is linked to a DNA binding ligand such as an intercalator compound are well known in the art and any convenient method can be used. Representative intercalating agents include mono-or bis-azido aminoalkyl methidium or ethidium compounds, ethidium monoazide ethidium diazide, ethidium dimer azide (Mitchell et al., J. Am. Chem. Soc., 104:4265 (1982))), 4-azido-7-chloroquinoline, 2-azidofluorene, 4′-aminomethyl-4,5′-dimethylangelicin, 4′-aminomethyl-trioxsalen (4′aminomethyl-4,5′,8-trimethyl-psoralen), 3-carboxy-5- or -8-amino- or -hydroxy-psoralen. A specific nucleic acid binding azido compound has been described by Forster et al., Nucleic Acid Res., 13:745 (1985). Other useful photoreactable intercalators are the furocoumarins which form (2+2) cycloadducts with pyrimidine residues. Alkylating agents also can be used as the DNA binding ligand, including, for example, bis-chloroethylamines and epoxides or aziridines, e.g., aflatoxins, polycyclic hydrocarbon epoxides, mitomycin and norphillin A. Particularly useful photoreactive forms of intercalating agents are the azidointercalators. Their reactive nitrenes are readily generated at long wavelength ultraviolet or visible light and the nitrenes of arylazides prefer insertion reactions over their rearrangement products (White et al., Meth Enzymol., 46:644 (1977)).

The probe may also be modified for use in a specific format such as the addition of 10-100 T residues for reverse dot blot or the conjugation to bovine serum albumin or immobilization onto magnetic beads.

When detecting hybridization by an indirect detection method, a detectably labeled second probe(s) can be added after initial hybridization between the probe and the target or during hybridization of the probe and the target. Optionally, the hybridization conditions may be modified after addition of the secondary probe. After hybridization, unhybridized secondary probe can be separated from the initial probe, for example, by washing if the initial probe is immobilized on a solid support. In the case of a solid support, detection of label bound to locations on the support indicates hybridization of a target nucleotide sequence in the sample to the probe.

The detectably labeled secondary probe can be a specific probe. Alternatively, the detectably labeled probe can be a degenerate probe, e.g., a mixture of sequences such as whole genomic DNA essentially as described in U.S. Pat. No. 5,348,855. In the latter case, labeling can be accomplished with intercalating dyes if the secondary probe contains double stranded DNA. Preferred DNA-binding ligands are intercalator compounds such as those described above.

A secondary probe also can be a library of random nucleotide probe sequences. The length of a secondary probe should be decided in view of the length and composition of the primary probe or the target nucleotide sequence on the solid support that is to be detected by the secondary probe. Such a probe library is preferably provided with a 3′ or 5′ end labeled with photoactivatable reagent and the other end loaded with a detection reagent such as a fluorophore, enzyme, dye, luminophore, or other detectably known moiety.

The particular sequence used in making the labeled nucleic acid can be varied. Thus, for example, an amino-substituted psoralen can first be photochemically coupled with a nucleic acid, the product having pendant amino groups by which it can be coupled to the label, i.e., labeling is carried out by photochemically reacting a DNA binding ligand with the nucleic acid in the test sample. Alternatively, the psoralen can first be coupled to a label such as an enzyme and then to the nucleic acid.

Advantageously, the DNA binding ligand is first combined with label chemically and thereafter combined with the nucleic acid probe. For example, since biotin carries a carboxyl group, it can be combined with a furocoumarin by way of amide or ester formation without interfering with the photochemical reactivity of the furocoumarin or the biological activity of the biotin. Aminomethylangelicin, psoralen and phenanthridium derivatives can similarly be linked to a label, as can phenanthridium halides and derivatives thereof such as aminopropyl methidium chloride (Hertzberg et al, J. Amer. Chem. Soc., 104:313 (1982)). Alternatively, a bifunctional reagent such as dithiobis succinnidyl propionate or 1,4-butanediol diglycidyl ether can be used directly to couple the DNA binding ligand to the label where the reactants have alkyl amino residues, again in a known manner with regard to solvents, proportions and reaction conditions. Certain bifunctional reagents, possibly glutaraldehyde may not be suitable because, while they couple, they may modify nucleic acid and thus interfere with the assay. Routine precautions can be taken to prevent such difficulties.

Also advantageously, the DNA binding ligand can be linked to the label by a spacer, which includes a chain of up to about 40 atoms, preferably about 2 to 20 atoms, including, but not limited to, carbon, oxygen, nitrogen and sulfur. Such spacer can be the polyfunctional radical of a member including, but not limited to, peptide, hydrocarbon, polyalcohol, polyether, polyamine, polyimine and carbohydrate, e.g., -glycyl-glycyl-glycyl- or other oligopeptide, carbonyl dipeptides, and omega-amino-alkane-carbonyl radical or the like. Sugar, polyethylene oxide radicals, glyceryl, pentaeyritol, and like radicals also can serve as spacers. Spacers can be directly linked to the nucleic acid-binding ligand and/or the label, or the linkages may include a divalent radical of a coupler such as dithiobis succinimidyl propionate, 1,4-butanediol diglycidyl ether, a diisocyanate, carbodiimide, glyoxal, glutaraldehyde, or the like.

Secondary probe for indirect detection of hybridization can be also detected by energy transfer such as in the “beacon probe” method described by Tyagi and Kramer, Nature Biotech., 14:303-309 (1996) or U.S. Pat. Nos. 5,119,801 and 5,312,728 to Lizardi et al. Any FRET detection system known in the art can be used in the present method. For example, the AlphaSereen™ system can be used. AlphaScreen technology is an “Amplified Luminescent Proximity Homogeneous Assay” method. Upon illumination with laser light at 680 nm, a photosensitizer in the donor bead converts ambient oxygen to singlet-state oxygen. The excited singlet-state oxygen molecules diffuse approximately 250 nm (one bead diameter) before rapidly decaying. If the acceptor bead is in close proximity of the donor bead, by virtue of a biological interaction, the singlet-state oxygen molecules reacts with chemiluminescent groups in the acceptor beads, which immediately transfer energy to fluorescent acceptors in the same bead. These fluorescent acceptors shift the emission wavelength to 520-620 nm. The whole reaction has a 0.3 second half-life of decay, so measurement can take place in time-resolved mode. Other exemplary FRET donor/acceptor pairs include Fluorescein (donor) and tetramethylrhodamine (acceptor) with an effective distance of 55 Å; IAEDANS (donor) and Fluorescein (acceptor) with an effective distance of 46 Å; and Fluorescein (donor) and QSY-7 dye (acceptor) with an effective distance of 61 Å (Molecular Probes).

Quantitative assays for nucleic acid detection also can be performed according to the present invention. The amount of secondary probe bound to a microarray spot can be measured and can be related to the amount of nucleic acid target which is in the sample. Dilutions of the sample can be used along with controls containing known amount of the target nucleic acid. The precise conditions for performing these steps will be apparent to one skilled in the art. In microarray analysis, the detectable label can be visualized or assessed by placing the probe array next to x-ray film or phosphoimagers to identify the sites where the probe has bound. Fluorescence can be detected by way of a charge-coupled device (CCD) or laser scanning.

Test Samples

Any suitable samples, including samples of human, animal, or environmental (e.g., soil or water) origin, can be analyzed using the present method. Test samples can include body fluids, such as urine, blood, semen, cerebrospinal fluid, pus, amniotic fluid, tears, or semisolid or fluid discharge, e.g., sputum, saliva, lung aspirate, vaginal or urethral discharge, stool or solid tissue samples, such as a biopsy or chorionic villi specimens. Test samples also include samples collected with swabs from the skin, genitalia, or throat.

Test samples can be processed to isolate nucleic acid by a variety of means well known in the art (See generally, Ausubel (Ed.) Current Protocols in Molecular Biology, 2. Preparation and Analysis of DNA and 4. Preparation and Analysis of RNA, John Wiley & Sons, Inc. (2000)). It will be apparent to those skilled in the art that target nucleic acids can be RNA or DNA that may be in form of direct sample or purified nucleic acid or amplicons.

Purified nucleic acids can be extracted from the aforementioned samples and may be measured spectraphotometrically or by other instrument for the purity. For those skilled in the art of nucleic acid amplification, amplicons are obtained as end products by various amplification methods such as PCR (Polymerase Chain Reaction, U.S. Pat. Nos. 4,683,195, 4,683,202, 4,800,159 and 4,965,188), NASBA (Nucleic Acid Sequence Based Amplification, U.S. Pat. No. 5,130,238), TMA (Transcription Mediated Amplification) (Kwoh et al., Proc. Natl. Acad Sci., USA, 86:1173-1177 (1989)), SDA (Strand Displacement Amplification, described by Walker et al., U.S. Pat. No. 5,270,184), tSDA (thermophilic Strand Displacement Amplification (U.S. Pat. No. 5,648,211 and Euro. Patent No. EP 0 684315), SSSR (Self-Sustained Sequence Replication) (U.S. Pat. No. 6,156,508).

In a specific embodiment, a sample of human origin is assayed. In yet another specific embodiment, a sputum, urine, blood, tissue section, food, soil or water sample is assayed.

Kits

The present probes can be packaged in a kit format, preferably with an instruction for using the probes to detect a target gene. The components of the kit are packaged together in a common container, typically including written instructions for performing selected specific embodiments of the methods disclosed herein. Components for detection methods, as described herein, may optionally be included in the kit, for example, a second probe, and/or reagents and means for carrying out label detection (e.g., radiolabel, enzyme substrates, antibodies, etc., and the like).

### F. EXAMPLES

### Example 1

### Probe Designs

Various genome sequences of SARS-CoV are available (See e.g., Table 6).

TABLE 6
Genome sequences of SARS coronaviruse currently obtained (as of 5/2/2003)

| | | | | Number | | |
|---|---|---|---|---|---|---|
| | Source of | Submitting | | of N in | Length | |
| | SARS | Country | GenBank | the | of the | Percentage |
| ID | coronaviruse | (Area) | Acc | sequence | genome | of N |
| SARS_BJ01 | Beijing, | China | AY278488 | 900 | 28920 | 3.11% |
| | China | | | | | |
| SARS_BJ02 | Beijing, | China | AY278487 | 300 | 29430 | 1.02% |
| | China | | | | | |
| SARS_BJ03 | Beijing, | China | AY278490 | 607 | 29291 | 2.07% |
| | China | | | | | |
| SARS_GZ01 | Guangzhou, | China | AY278489 | 1007 | 29429 | 3.42% |
| | China | | | | | |
| SARS_BJ04 | Beijing, | China | AY279354 | 2502 | 24774 | 10.10% |
| | China | | | | | |
| SARS_{—} | Hong Kong, | Hong Kong, | AY278554 | 0 | 29736 | 0.00% |
| CUHK-W1 | China | China | | | | |
| SARS_HKU-3 | Hong Kong, | Hong Kong, | AY278491 | 0 | 29742 | 0.00% |
| 9849 | China | China | | | | |
| SARS_Urbani | Vietnam | U.S. | AY278741 | 0 | 29727 | 0.00% |
| SARS_TOR2 | Toronto, | Canada | AY274119 | 0 | 29736 | 0.00% |
| | Canada | | | | | |

The sizes of the nine genomes shown in Table 6 are very similar. The five genomes submitted by China contain various levels of unidentified nucleotides (N).

The following Table 7 shows similarities or homologies among the nine genomes of SARS coronaviruse.

TABLE 7
Comparison of similarities between the nine genomes of SARS coronaviruse

| | BJ01 | BJ02 | BJ03 | GZ01 | BJ04 | CUHK-W1 | HKU-39849 | Urbani | TOR2 |
|---|---|---|---|---|---|---|---|---|---|
| BJ01 | | [symbol] | [symbol] | [symbol] | 91 | [symbol] | [symbol] | [symbol] | [symbol] |
| BJ02 | [symbol] | | [symbol] | 94 | [symbol] | [symbol] | [symbol] | [symbol] | [symbol] |
| BJ03 | [symbol] | [symbol] | | [symbol] | [symbol] | [symbol] | [symbol] | [symbol] | [symbol] |
| GZ01 | [symbol] | 94 | [symbol] | | 91 | [symbol] | [symbol] | [symbol] | [symbol] |
| BJ04 | 91 | 88 | [symbol] | 91 | | [symbol] | 89 | 89 | 89 |
| CUHK-W1 | [symbol] | [symbol] | [symbol] | [symbol] | [symbol] | | [symbol] | [symbol] | [symbol] |
| HKU-39849 | [symbol] | [symbol] | [symbol] | [symbol] | [symbol] | [symbol] | | [symbol] | [symbol] |
| Urbani | [symbol] | [symbol] | [symbol] | [symbol] | [symbol] | [symbol] | [symbol] | | [symbol] |
| TOR2 | [symbol] | [symbol] | [symbol] | [symbol] | [symbol] | [symbol] | [symbol] | [symbol] | |

The similarity of the nine genomes of SARS coronaviruse were compared. The numbers shown in the Table 7 represent the percentage of similarity between two genomes. Each number in Table 7 equals to the number of the same bases in two genomes divided by the total number of bases (about 30,000 bases) compared and then timed by 100.

Table 7 shows that the different genomes of SARS coronaviruse are highly similar to each other except BJ04. The similarity lower than 99% is caused by the presence of N in the nucleotide sequence. If all the Ns in the nucleotide sequences from BJ01-BJ04 and GZ01 are considered as the same with other genome (this assumption is reasonable based on comparison of other part of the genomes), the nine genomes are 99% similar to each other.

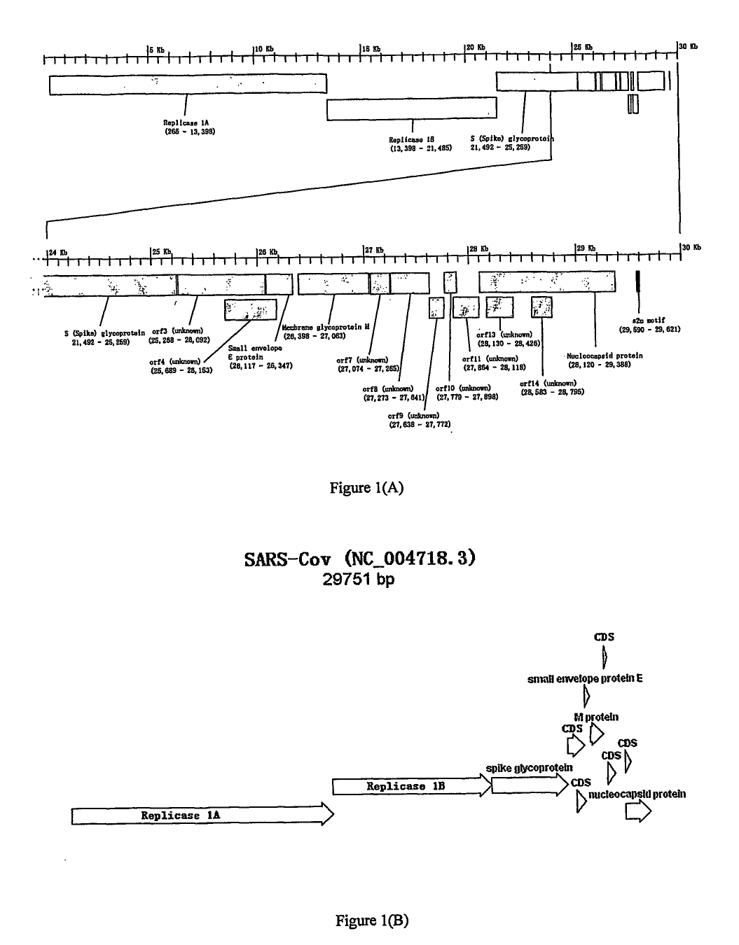

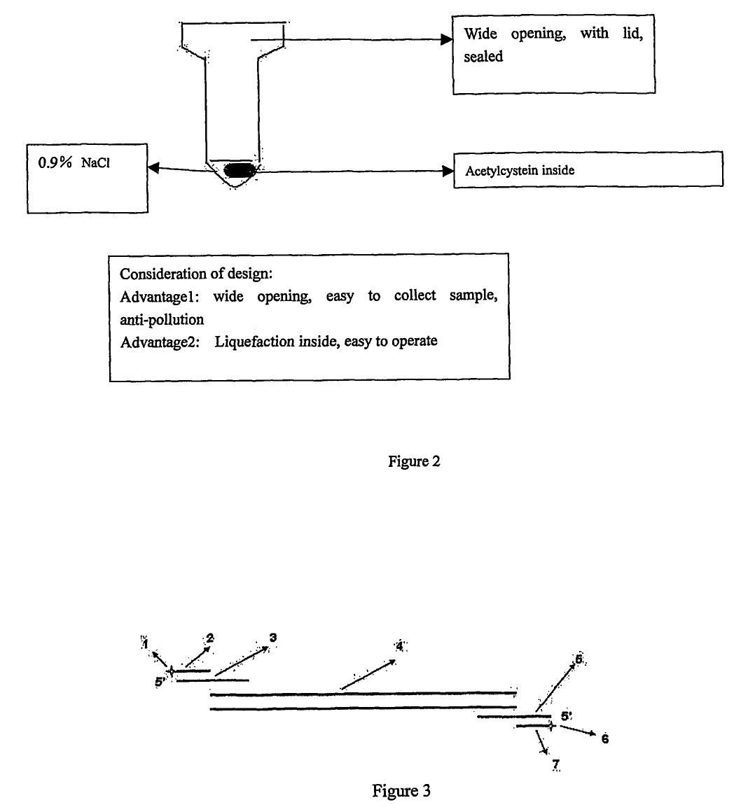

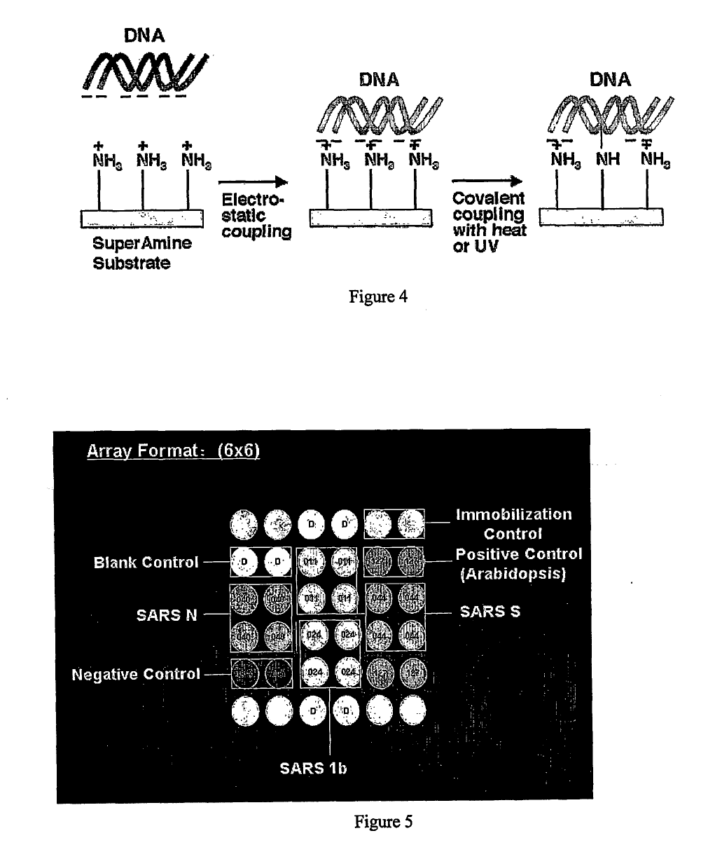

Since SARS coronaviruse is conservative as shown in Tables 6 and 7, nucleic acid based detection methods are rational. FIG. 1B indicates that detection of different parts of SARS coronaviruse genome at the same time can significantly increase the sensitivity and specificity of the detection method.

We have two overall designs. One design is to perform a multiplex PCR for different parts of SARS coronaviruse genome and use PCR products as probes for detection. The second design is to perform a multiplex PCR for different parts of SARS coronaviruse genome and use a 70 mer oligonucleotides as probes for detection.

Target Gene Selection

Based on analysis of SARS coronaviruse genome, we selected three genes as target genes. These three genes are orf 1A and 1B polymerase proteins, spike protein, and nucleocapsid protein. We selected human housekeeping gene GAPD (glyceraldehyde 3-phosphate dehydrogenase) (GenBank Acc: NM_{—}002046) as a positive control for RNA isolation. We selected a gene (Arabidopsis) (GenBank Acc: AJ441252), which has no homology to nucleotide sequence of human and common pathogens, as incorporated positive control.

Design of Primers and Probes

The three proteins of SARS coronaviruse were analyzed and their conservative sequences were compared. According to the requirement of multiplex PCR, multiple pairs of primers, which have similar Tm values and are 1.5 Kb in distance, and have amplified products between 200 to 900 bp, were designed based on the conservative sequence between different genomes. In addition, multiple non-overlapping oligonucleotides (70 mer) were designed based on amplified product of each pair of primers. These primers and probes were compared with the most updated NCBI nucleic acid non-redundant nucleotide database using BLASTN, and the specificities of the probes and primers were assured.

### Example 2

### Process for Pretreatment of Blood Sample

Pretreatment of blood sample involves relatively complicated processes. However, considering the relative low concentration SARS virus in serum reported, pretreatment described herein can effectively enrich lymphocytes from about 2 ml of the whole blood in order to increase the chances of detection.

1. Sample Collection and Transfer

1) Samples collected from patients in the hospital room are put in a first transfer window. The door of the window is then closed and locked.

2) The samples are then transferred into a second transfer window. The samples are recorded in a notebook and three bar code labels are printed. The samples are tested for conventional detection and transferred into a pretreatment transfer window.

2. Use of Biosafe Cabinet

1) Hospital personnel for performing pretreatment process enters the pretreatment room and close the door. The biosafe cabinet is then turned on. The fan of the cabinet and light are then automatically turned on.

2) The indicator lights for power switch, air speed switch, and work light switch are checked for normal operation. The indicator light for air selection switch is checked as off status. Abnormal or unusual operation is reported.

3) The indicator light for alarm switch will make an alarm sound which indicates normal status of the biosafe cabinet after self-testing. Fifteen minutes later, the alarm sound from the indicator light for alarm switch is stopped and the process in the biosafe cabinet can be started.

4) The process in the cabinet cannot be started if the alarm sound continues or the process has to be stopped if there is an alarm sound during the process. The incident has to be reported immediately.

5) After the biosafe cabinet operates normally, samples are taken from the second transfer window and placed in the cabinet. The transfer window top is cleaned by wiping with 75% alcohol and spraying with 0.5% peracetic acid. The door for the transfer window is then closed and locked.

6) The complete process of sample pretreatment is then performed in the biosafe cabinet.

3. Serum Isolation

1) Blood (1.8 ml) with anticoagulant is centrifuged for 10 minutes at 3,500 rpm. The top layer is marked with a marker pen.

2) The top layer serum (about 1.0 ml) is then collected and put into a 1.5 ml sterile Eppendorf centrifuge tube.

3) The Eppendorf centrifuge tube is labeled with the bar code (marked as “P”) and labeled with a sequence number.

4) The sample is then recorded in a notebook.

5) The centrifuge tube containing the serum sample is put in a specialized sample box and stored at −80° C. The outside of the sample box is labeled with SARS, serum and range of sample numbers.

4. Isolation of Blood Cells

1) Lymphocyte isolation solution (3.6 ml) is added to a 10 ml centrifuge tube.

2) Sterile physiological saline (a volume equal to the serum taken out in the centrifuge tube described above) is added to the centrifuge tube containing the blood cells. The blood cells are resuspended in saline using Pasteur pipette.

3) The resuspended blood cells are slowly loaded on top of the lymphocyte 20 isolation solution and centrifuged for 20 minutes at 1,500 rpm.

4) The cells located between the layers are collected and put in a 1.5 ml sterile Eppendorf centrifuge tube, which is then centrifuged for 5 minutes at 10,000 rpm to spin down the cells. The supernatant is withdrawn.

5) The tube containing the cell pellet is then labeled with the bar code (marked “C”) and labeled with a sequence number.

6) The sample is recorded in a notebook.

7) The centrifuge tube containing the blood cell sample is put in a specialized sample box and stored at -80° C. The outside of the sample box is labeled with SARS, blood cells, and range of sample numbers.

8) The glass face plate of the biosafe cabinet is then opened. The bench surface and other surfaces in the biosafe cabinet are then sterilized by wiping with 70% alcohol and spraying 0.5% peracetic acid.

9) After cleaning, the glass face plate is closed. The ultraviolet light is placed inside the cabinet and turned on for 15 minutes.

10) The power switch of the biosafe cabinet is turned off before leaving the sample pretreatment room.

5. Mattes Needing Attention

1) The lymphocyte isolation solution should not be used immediately after being taken out of the refrigerator. The solution should be used after its temperature reaches room temperature and the solution is mixed well.

2) The whole isolation process should be performed at 18-28° C. Too high or too low temperature can impact on the quality of isolation process.

3) The pipette tips, Eppendorf centrifuge tubes, gloves, and disposed reagents or liquids should be discarded in a waist tank (containing 0.5% peracetic acid). Everything in the waster tank should be treated at high pressure after experiment and then discarded.

4) 0.5% of peracetic acid is prepared by diluting 32 ml of 16% of peracetic acid in H₂O to make a final volume of 1,000 ml.

### Example 3

### Process for Extracting RNA Using QIAamp Viral PNA Kit

The following procedures are used in RNA preparation:

1. Pipet 560 μl of prepared Buffer AVL containing Carrier RNA into a 1.5-ml microcentrifuge tube. If the sample volume is larger than 140 μl, increase the amount of Buffer AVL/Carrier RNA proportionally (e.g., a 280-μl sample will require 1120 μl Buffer AVL/Carrier RNA).

2. Add 140 μl plasma, serum, urine, cell-culture supernatant, or cell-free body fluid to the Buffer AVL/Carrier RNA in the microcentrifuge tube. Mix by pulse-vortexing for 15 sec. To ensure efficient lysis, it is essential that the sample is mixed thoroughly with Buffer AVL to yield a homogeneous solution. Frozen samples that have only been thawed once can also be used.

3. Incubate at room temperature (15-25° C.) for 10 min. Viral particle lysis is complete after lysis for 10 min at room temperature. Longer incubation times have no effect on the yield or quality of the purified RNA. Potentially infectious agents and RNases are inactivated in Buffer AVL.

4. Briefly centrifuge the 1.5-ml microcentrifuge tube to remove drops from the inside of the lid.

5. Add 560 μl of ethanol (96-100%) to the sample, and mix by pulse-vortexing for 15 sec. After mixing, briefly centrifuge the 1.5-ml microcentrifuge tube to remove drops from inside the lid. Only ethanol is preferred since other alcohols may result in reduced RNA yield and purity. If the sample volume is greater than 140 μl, increase the amount of ethanol proportionally (e.g., a 280-μl sample will require 1120 μl of ethanol). In order to ensure efficient binding, it is essential that the sample is mixed thoroughly with the ethanol to yield a homogeneous solution.

6. Carefully apply 630 μl of the solution from step 5 to the QIAamp spin column (in a 2-ml collection tube) without wetting the rim. Close the cap, and centrifuge at 6000×g (8000 rpm) for 1 min. Place the QIAamp spin column into a clean 2-ml collection tube, and discard the tube containing the filtrate. Close each spin column in order to avoid cross-contamination during centrifugation. Centrifugation is performed at 6,000×g (8,000 rpm) in order to limit microcentrifuge noise. Centrifugation at full speed will not affect the yield or purity of the viral RNA. If the solution has not completely passed through the membrane, centrifuge again at a higher speed until all of the solution has passed through.

7. Carefully open the QIAamp spin column, and repeat step 6. If the sample volume is greater than 140 μl, repeat this step until all of the lysate has been loaded onto the spin column.

8. Carefully open the QIAamp spin column, and add 500 μl of Buffer AW1. Close the cap, and centrifuge at 6,000×g (8,000 rpm) for 1 min. Place the QIAamp spin column in a clean 2-ml collection tube (provided), and discard the tube containing the filtrate. It is not necessary to increase the volume of Buffer AW1 even if the original sample volume was larger than 140 μl.

9. Carefully open the QIAamp spin column, and add 500 μl of Buffer AW2. Close the cap and centrifuge at full speed (20,000×g; 14,000 rpm) for 3 min. Continue directly with step 10, or to eliminate any chance of possible Buffer AW2 carryover, perform step 9a, and then continue with step 10. Note: Residual Buffer AW2 in the eluate may cause problems in downstream applications. Some centrifuge rotors may vibrate upon deceleration, resulting in flow-through, containing Buffer AW2, contacting the QIAamp spin column. Removing the QIAamp spin column and collection tube from the rotor may also cause flowthrough to come into contact with the QIAamp spin column. In these cases, the optional step 9a should be performed.

9a. (Optional): Place the QIAamp spin column in a new 2-ml collection tube (not provided), and discard the old collection tube with the filtrate. Centrifuge at full speed for 1 min.

10. Place the QIAamp spin column in a clean 1.5-ml microcentrifuge tube (not provided). Discard the old collection tube containing the filtrate. Carefullly open the QIAamp spin column and add 60 μl of Buffer AVE equilibrated to room temperature. Close the cap, and incubate at room temperature for 1 min. Centrifuge at 6,000×g (8,000 rpm) for 1 min. A single elution with 60 μl of Buffer AVE is sufficient to elute at least 90% of the viral RNA from the QIAamp spin column. Performing a double elution using 2×40 μl of Buffer AVE will increase yield by up to 10%. Elution with volumes of less than 30 μl will lead to reduced yields and will not increase the final concentration of RNA in the eluate. Viral RNA is stable for up to one year when stored at −20° C. or −70° C.

The following are further information pertaining to the above procedures:
Equilibrate samples to room temperature (15-25° C.).
Equilibrate Buffer AVE to room temperature for elution in step 10.
Check whether Buffer AW1, Buffer AW2, and Carrier RNA have been prepared according to the instructions on pages 14-15.
Redissolve precipitate in Buffer AVL/Carrier RNA by heating, if necessary, and cool to room temperature before use.
All centrifugation steps are carried out at room temperature.

### Example 4

### An exemplary Array format of SARS-CoV Detection Chin

FIG. 5 illustrates an exemplary array format of SARS-CoV detection chip.

Immobilization control is an oligo-probe that is labeled by a fluorescent dye HEX on its end and does not participate in any hybridization reaction when a sample containing or suspected of containing of a SARS-CoV is contacted with the chip.

Positive control(Arabidopsis) is an oligo-probe designed according to an Arabidopsis (one kind of model organism) gene and does not participate in any hybridization reaction when a sample containing or suspected of containing of a SARS-CoV is contacted with the chip. During hybridization reaction, target probes that can hybridize with this positive control perfectly are added into the hybridization solution. Signals of the positive control can be applied to monitor the hybridization reaction.

Negative control is an oligo-probe that does not participate in any hybridization reaction when a sample containing or suspected of containing of a SARS-CoV is contacted with the chip.

Blank Control is DMSO solution spot. It is used for monitoring arraying quality.

SARS probes are 011, 024, 040 and 044 probes.

### Example 5

### SARS-CoV Detection From a SARS Patient Blood Sample (Sample No. 3)

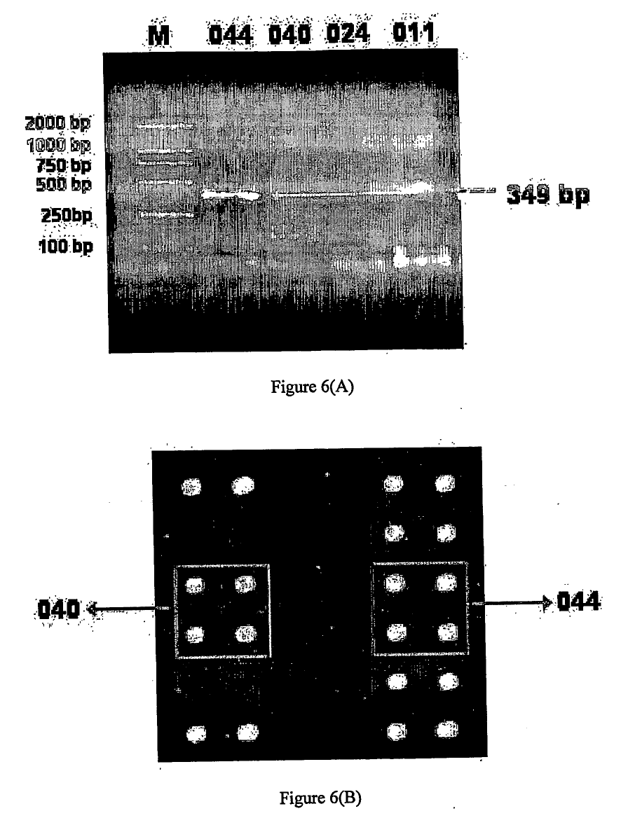

FIGS. 6A and 6B illustrate SARS-CoV detection from a SARS patient blood sample (sample No. 3). Lymphocytes were isolated from 3# SARS patient blood sample. RNA from lymphocytes was extracted by QIAamp Kit. RT-nest PCR was performed using RNA extracted above as templates. 044 RT-nest PCR result was good and hybridization result was good too. 040 RT-nest PCR result was poor but hybridization result was good. It shows that the chip-hybridization method is sensitive and specific.

### Example 6

### SARS-CoV Detection From a SARS Patient Blood Sample (Sample No. 4

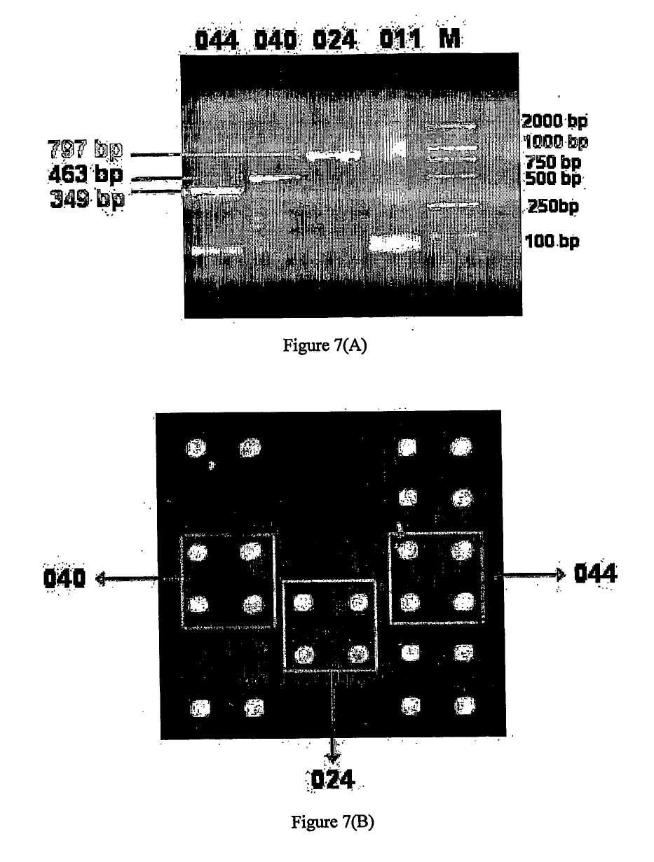

FIGS. 7A and 7B illustrate SARS-CoV detection from a SARS patient blood sample (sample No. 4). Lymphocytes were isolated from 4# SARS patient blood sample. RNA from lymphocytes was extracted by QIAamp Kit. RT-nest PCR was performed using RNA extracted above as templates. 024, 040 and 044 RT-nest PCR results were good and hybridization results were good too.

### Example 7

### SARS-CoV Detection From a SARS Patient Sputum Sample (Sample No. 5)

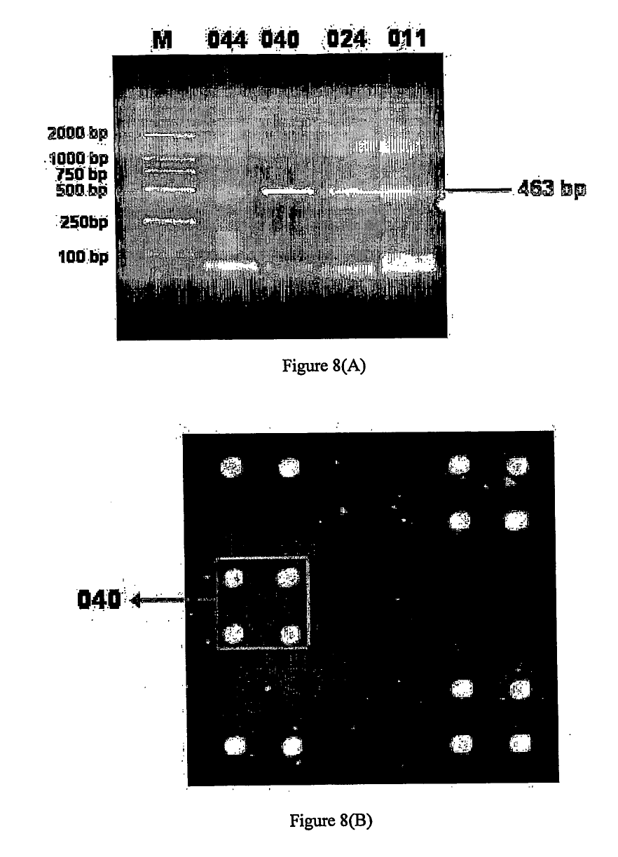

FIG. 8 illustrates SARS-CoV detection from a SARS patient sputum sample (sample No. 5). RNA from 5# SARS patient sputum sample was extracted by QIAamp Kit. RT-nest PCR was performed using RNA extracted above as templates. 040 RT-nest PCR result was good and hybridization result was good too.

### Example 8

### SARS-CoV Detection From a SARS Patient Sputum Sample (Sample No. 6)

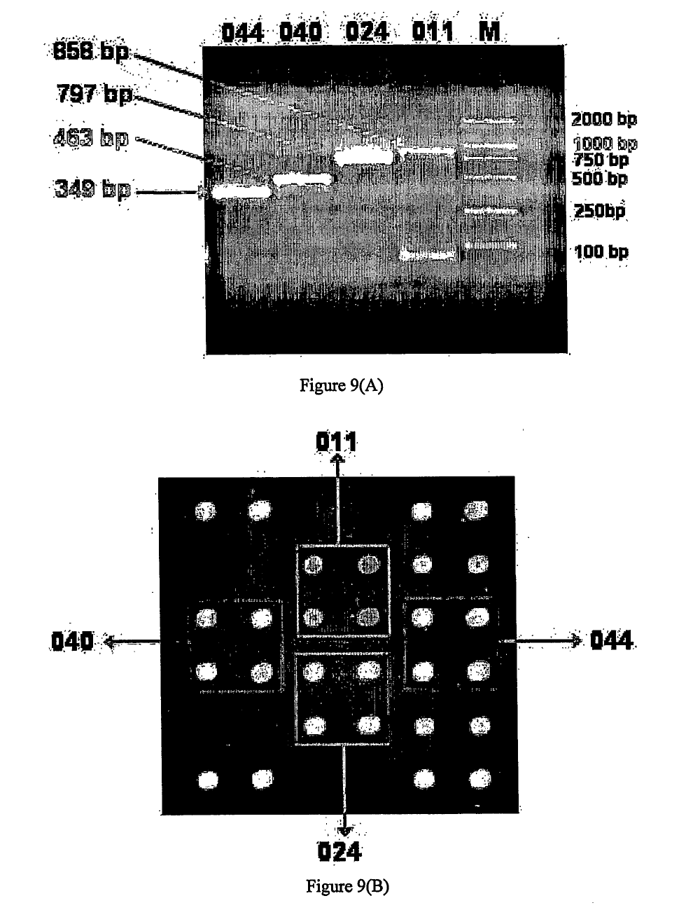

FIG. 9 illustrates SARS-CoV detection from a SARS patient sputum sample (sample No. 6). RNA from 6# SARS patient sputum sample was extracted by QIAamp Kit. RT-nest PCR was performed using RNA extracted above as templates. All probes RT-nest PCR results were good and hybridization results were good too.

### Example 9

### Another Exemplary Array Format of SARS-CoV Detection Chip

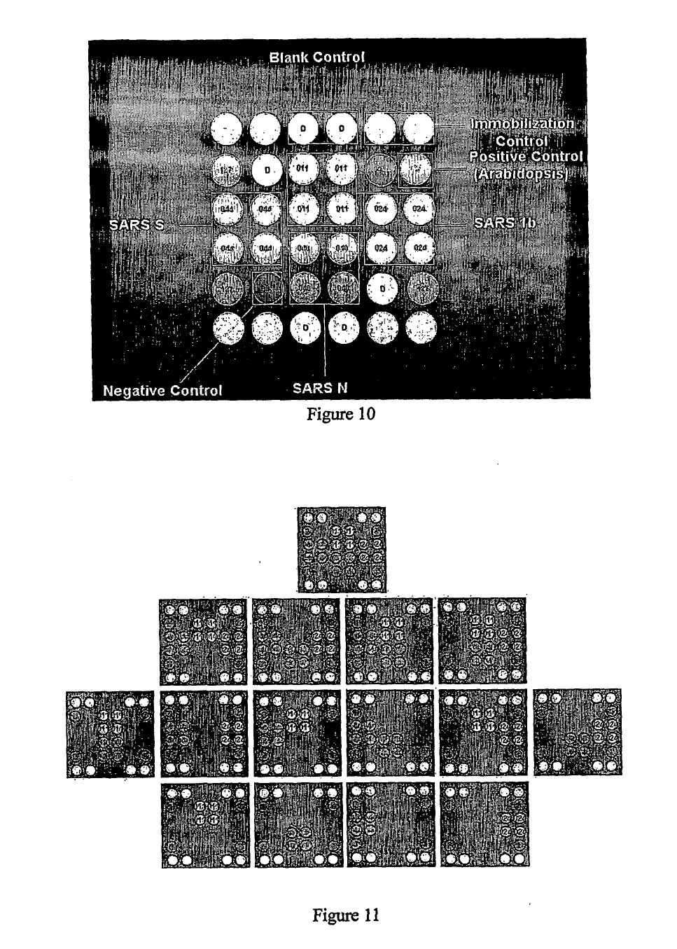

FIG. 10 illustrates another exemplary array format of SARS-CoV detection chip.

Immobilization control is an oligo-probe that is labeled by a fluorescent dye HEX on its end and does not participate in any hybridization reaction when a sample containing or suspected of containing of a SARS-CoV is contacted with the chip.

Positive control (Arabidopsis) is an oligo-probe designed according to an Arabidopsis (one kind of model organism) gene and does not participate in any hybridization reaction when a sample containing or suspected of containing of a SARS-CoV is contacted with the chip. During hybridization reaction, target probes that can hybridize with this positive control perfectly are added into the hybridization solution. Signals of the positive control can be applied to monitor the hybridization reaction.

Negative control is an oligo-probe that does not participate in any hybridization reaction when a sample containing or suspected of containing of a SARS-CoV is contacted with the chip.

Blank Control is DMSO solution spot. It is used for monitoring arraying quality.

SARS probes are 011, 024, 040 and 044 probes.

### Example 10

### All Possible Positive Results on the SARS SARS-CoV Detection Chip Illustrated in FIG. 10

FIG. 11 illustrates all possible positive results on the SARS SARS-CoV detection chip illustrated in FIG. 10.

There are four sets probes on chips for detecting SARS virus: probe 011, probe 024, probe 040 and probe 044.

The first line gives the positive result (1) by signals appearing on all four sets of probes: 011+024+040+044.

The second line gives all the possible positive results (4) by signals appearing on three sets probes: 011+024+044, 024+040+044, 011+040+044, 011+024+040.

The third line gives all the possible positive results (6) by signals appearing on two sets probes: 011+040, 024+044, 011+044, 040+044, 011+024, 024+040.

The fourth line gives all the possible positive results (4) by signals appearing on only one set probes: 011, 024, 040, 044.

The above examples are included for illustrative purposes only and are not intended to limit the scope of the invention. Many variations to those described above are possible. Since modifications and variations to the examples described above will be apparent to those of skill in this art, it is intended that this invention be limited only by the scope of the appended claims.

1. A chip for assaying for a coronaviruse causing the severe acute respiratory syndrome (SARS-CoV), which chip comprises a support suitable for use in nucleic acid hybridization having immobilized thereon at least two oligonucleotide probes complementary to at least two different nucleotide sequences of SARS-CoV genome, each of said two different nucleotide sequences comprising at least 10 nucleotides.

2. The chip of claim 1, wherein the at least two different nucleotide sequences of SARS-CoV genome comprises:
a) a nucleotide sequence of at least 10 nucleotides located within a conserved region of SARS-CoV genome and a nucleotide sequence of at least 10 nucleotides located within a variable region of SARS-COV genome; or
b) a nucleotide sequence of at least 10 nucleotides located within a structural protein coding gene of SARS-CoV genome and a nucleotide sequence of at least 10 nucleotides located within a non-structural protein coding gene of SARS-CoV genome.

3. The chip of claim 1, which further comprises:
a) at least one of the following three oligonucleotide probes: an immobilization control probe that is labeled and does not participate in any hybridization reaction when a sample containing or suspected of containing of a SARS-CoV is contacted with the chip, a positive control probe that is not complementary to any SARS-CoV sequence but is complementary to a non-SARS-CoV-sequence contained in the sample and a negative control probe that is not complementary to any nucleotide sequence contained in the sample; and
b) a blank spot.

4. The chip of claim 1, which comprises at least two oligonucleotide probes complementary to two different nucleotide sequences of at least 10 nucleotides, respectively, located within a conserved region of SARS-CoV genome, located within a structural protein coding gene of SARS-CoV genome or located within a non-structural protein coding gene of SARS-CoV genome.

5. The chip of claim 1, wherein:
a) the conserved region of SARS-CoV genome is a region located within the Replicase 1A or 1B gene or the Nucleocapsid N) gene of SARS-CoV;
b) the structural protein coding gene of SARS-CoV genome is a gene encoding the Spike glycoprotein (S), the small envelope protein (E) or the Nucleocapsid protein (N); or
c) the non-structural protein coding gene of SARS-CoV genome is a gene encoding the Replicase 1A or 1B.

6. The chip of claim 1, wherein the variable region of SARS-CoV genome is a region located within the Spike glycoprotein (S) gene of SARS-CoV.

7. The chip of claim 1, which comprises at least two of the following four oligonucleotide probes: two oligonucleotide probes complementary to two different nucleotide sequences of at least 10 nucleotides located within the Replicase 1A or 1B gene of SARS-CoV, an oligonucleotide probe complementary to a nucleotide sequence of at least 10 nucleotides located within the N gene of SARS-CoV and an oligonucleotide probe complementary to a nucleotide sequence of at least 10 nucleotides located within the S gene of SARS-CoV.

8. The chip of claim 7, wherein one of the two different nucleotide sequences located within the Replicase 1A or 1B gene of SARS-CoV comprises a nucleotide sequence that:
a) hybridizes, under high stringency, with a Replicase 1A or 1B nucleotide sequence, or a complementary strand thereof, that is set forth in Table 3; or
b) has at least 90% identity to a Replicase 1A or 1B nucleotide sequence comprising a nucleotide sequence, or a complementary strand thereof, that is set forth in Table 3.

9. The chip of claim 8, wherein one of the two different nucleotide sequences located within the Replicase 1A or 1B gene of SARS-CoV comprises a nucleotide sequence that is set forth in Table 3.

10. The chip of claim 7, wherein the nucleotide sequence located within the N gene of SARS-CoV comprises a nucleotide sequence that:
a) hybridizes, under high stringency, with a N nucleotide sequence, or a complementary strand thereof, that is set forth in Table 3; or
b) has at least 90% identity to a N nucleotide sequence comprising a nucleotide sequence, or a complementary strand thereof, that is set forth in Table 3.

11. The chip of claim 1O, wherein the nucleotide sequence located within the N gene of SARS-CoV comprises a nucleotide sequence that is set forth in Table 3.

12. The chip of claim 7, wherein the nucleotide sequence located within the S gene of SARS-CoV comprises a nucleotide sequence that:
a) hybridizes, under high stringency, with a S nucleotide sequence, or a complementary strand thereof, that is set forth in Table 3; or
b) has at least 90% identity to a S nucleotide sequence comprising a nucleotide sequence, or a complementary strand thereof, that is set forth in Table 3.

13. The chip of claim 12, wherein the nucleotide sequence located within the S gene of SARS-CoV comprises a nucleotide sequence that is set forth in Table 3.

14. The chip of claim 3, wherein the label of the immobilization control probe is selected from the group consisting of a chemical, an enzymatic, an immunogenic, a radioactive, a fluorescent, a luminescent and a FRET label.

15. The chip of claim 3, wherein the non-SARS-CoV-sequence is spiked in the sample to be assayed.

16. The chip of claim 15, wherein the spiked non-SARS-CoV-sequence is a sequence of Arabidopsis origin.

17. The chip of claim 7, which comprises two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe that is labeled and does not participate in any hybridization reaction when a sample containing or suspected of containing of a SARS-CoV is contacted with the chip, a positive control probe that is not complementary to any SARS-CoV sequence but is complementary to a non-SARS-CoV-sequence contained in the sample and a negative control probe that is not complementary to any nucleotide sequence contained in the sample.

18. The chip of claim 17, which comprises multiple spots of the two oligonucleotide probes complementary to two different nucleotide sequences located within the Replicase 1B gene of SARS-CoV, the oligonucleotide probe complementary to a nucleotide sequence located within the N gene of SARS-CoV, the oligonucleotide probe complementary to a nucleotide sequence located within the S gene of SARS-CoV, the immobilization control probe, the positive control probe and the negative control probe.

19. The chip of claim 1, further comprising an oligonucleotide probe complementary to a nucleotide sequence of a coronaviruse not related to SARS-CoV.

20. The chip of claim 19, wherein the coronaviruse not related to the SARS is the Group I, II or III coronaviruse or is a coronaviruse that infects an avian species, an equine species, a canine species, a feline species, a porcine species, a calf species, a bovine species, a murine species, a puffinosis species, a rat species, a turkey species or a human species.

21. The chip of claim 3, wherein at least one of the oligonucleotide probe comprises, at its ′5 end, a poly dT region to enhance its immobilization on the support.

22. The chip of claim 1, wherein at least one of the oligonucleotide probes is complementary to a highly expressed nucleotide sequence of SARS-CoV genome.

23. The chip of claim 1, wherein the support comprises a surface that is selected from the group consisting of a silicon, a plastic, a glass, a ceramic, a rubber, and a polymer surface.

24. A method for assaying for a SARS-CoV in a sample, which methods comprises:
a) providing a chip of claim 1;
b) contacting said chip with a sample containing or suspected of containing a SARS-CoV nucleotide sequence under conditions suitable for nucleic acid hybridization; and
c) assessing hybrids formed between said SARS-CoV nucleotide sequence, if present in said sample, and said at least two oligonucleotide probes complementary to two different nucleotide sequences of SARS-CoV genome, respectively, to determine the presence, absence or amount of said SARS-CoV in said sample,
whereby detection of one or both said hybrids indicates the presence of said SARS-CoV in said sample.

25. A method for assaying for a SARS-CoV in a sample, which methods comprises:
a) providing a chip of claim 2;
b) contacting said chip with a sample containing or suspected of containing a SARS-CoV nucleotide sequence under conditions suitable for nucleic acid hybridization; and
c) assessing hybrids formed between said SARS-CoV nucleotide sequence, if present in said sample, and
i) said oligonucleotide probe complementary to a nucleotide sequence located within a conserved region of SARS-CoV genome and an oligonucleotide probe complementary to a nucleotide sequence located within a variable region of SARS-CoV genome, respectively; or
ii) said oligonucleotide probe complementary to a nucleotide sequence located within a structural protein coding gene of SARS-CoV genome and an oligonucleotide probe complementary to a nucleotide sequence located within a non-structural protein coding gene of SARS-CoV genome,
to determine the presence, absence or amount of said SARS-CoV in said sample,
whereby detection of one or both said hybrids indicates the presence of said SARS-CoV in said sample.

26. A method for assaying for a SARS-CoV in a sample, which methods comprises:
a) providing a chip of claim 3;
b) contacting said chip with a sample containing or suspected of containing a SARS-CoV nucleotide sequence under conditions suitable for nucleic acid hybridization; and
c) assessing:
(i) hybrids formed between said SARS-CoV nucleotide sequence, if present in the sample, and the oligonucleotide probe complementary to a nucleotide sequence within a conserved region of SARS-CoV genome and an oligonucleotide probe complementary to a nucleotide sequence located within a variable region of SARS-CoV genome, respectively;
(ii) a label comprised in the immobilization control probe, or a hybrid(s) involving the positive control probe and/or the negative control probe; and
(iii) a signal at said blank spot
to determine the presence, absence or amount of said SARS-CoV in a sample.

27. The method of claim 26, wherein the chip comprises two oligonucleotide probes complementary to two different nucleotide sequences located within the Replicase 1A or 1B gene of SARS-CoV, an oligonucleotide probe complementary to a nucleotide sequence located within the N gene of SARS-CoV, an oligonucleotide probe complementary to a nucleotide sequence located within the S gene of SARS-CoV, an immobilization control probe, a positive control probe and a negative control probe and the presence of the SARS-CoV is determined when:
a) a positive hybridization signal is detected using at least one of the two oligonucleotide probes complementary to two different nucleotide sequences located within the Replicase 1A or 1B gene of SARS-CoV, the oligonucleotide probe complementary to a nucleotide sequence located within the N gene of SARS-CoV and/or the oligonucleotide probe complementary to a nucleotide sequence located within the S gene of SARS-CoV;
b) a positive signal is detected from the immobilization control probe;
c) a positive hybridization signal is detected using the positive control probe;
d) a positive hybridization signal is not detected using the negative control probe; and
e) a positive hybridization signal is not detected at the blank spot.

28. The method of claim 27, wherein detecting a positive hybridization signal using at least one of the two oligonucleotide probes complementary to two different nucleotide sequences located within the Replicase 1A or 1B gene of SARS-CoV, or the oligonucleotide probe complementary to a nucleotide sequence located within the N gene of SARS-CoV, while not detecting a positive hybridization signal using the oligonucleotide probe complementary to a nucleotide sequence located within the S gene of SARS-CoV indicates mutation of the SARS-CoV.

29. The method of claim 24, which is used to positively identify SARS-CoV infected patients from a population of patients who have SARS-like symptoms.

30. The method of claim 24, wherein the chip of claim 19 is used and the method is used to positively identify SARS-CoV infected patients from patients who have been infected with a coronaviruse not related to the SARS.

31. The method of claim 24, wherein the chip of claim 22 is used and the method is used to diagnose early-stage SARS patients.

32. The method of claim 31, wherein the early-stage SARS patients have been infected with SARS-CoV from about less than one day to about three days.

33. The method of claim 24, wherein the SARS-CoV nucleotide sequence is a SARS-CoV RNA genomic sequence or a DNA sequence amplified from an extracted SARS-CoV RNA genomic sequence.

34. The method of claim 33, wherein the SARS-CoV RNA genomic sequence is extracted from a SARS-CoV infected cell using the QIAamp Viral RNA kit, the Chomczynsiki-Sacchi technique or TRIzol.

35. The method of claim 34, wherein the SARS-CoV RNA genomic sequence is extracted from a SARS-CoV infected cell using the QIAamp Viral RNA kit.

36. The method of claim 33, wherein the SARS-CoV RNA genomic sequence is extracted from a sputum or saliva sample, a lymphocyte of a blood sample.

37. The method of claim 33, wherein the SARS-CoV RNA genomic sequence is extracted from nasopharyngeal, oropharyngeal, tracheal, bronchaleolar lavage, pleural fluid, urine, stool, conjunctiva, tissue from human, mouse, dog, rat, cat, horse, avian, earth, water, air.

38. The method of claim 33, wherein the SARS-CoV RNA genomic sequence is amplified by PCR.

39. The method of claim 38, wherein a label is incorporated into the amplified DNA sequence during the PCR.

40. The method of claim 38, wherein the PCR comprises conventional, multiplex, nested PCR or RT-PCR.

41. The method of claim 38, wherein the PCR comprises a two-step nested PCR, the first step being a RT-PCR and the second step being a conventional PCR.

42. The method of claim 38, wherein the PCR comprises a one-step, multiplex RT-PCR using a plurality of 5′ and 3′ specific primers, each of the specific primers comprising a specific sequence complementary to its target sequence to be amplified and a common sequence, and a 5′ and a 3′ universal primer, the 5′ universal primer being complementary to the common sequence of the 5′ specific primers and the 3′ universal primer being complementary to the common sequence of the 3′ specific primers, and wherein in the PCR, the concentration of the 5′ and 3′ universal primers equals to or is higher than the concentration of the 5′ and 3′ specific primers, respectively.

43. The method of claim 42, wherein the 3′ universal primer and/or the 5′ universal primer is labeled.

44. The method of claim 43, wherein the label is a fluorescent label.

45. The method of claim 38, wherein the PCR comprises a multiplex nested PCR.

46. The method of claim 38, wherein the PCR is conducted using at least one of the following pairs of primers set forth in Table 4.

47. An oligonucleotide primer for amplifying a SARS-CoV nucleotide sequence, which oligonucleotide primer comprises a nucleotide sequence that:
a) hybridizes, under high stringency, with a target SARS-CoV nucleotide sequence, or a complementary strand thereof, that is set forth in Table 1; or
b) has at least 90% identity to a target SARS-CoV nucleotide sequence comprising a nucleotide sequence, or a complementary strand thereof, that is set forth in Table 1.

48. The primer of claim 47, which comprises DNA, RNA, PNA or a derivative thereof.

49. The primer of claim 47, which comprises a nucleotide sequence, or a complementary strand thereof, that is set forth in Table 1.

50. A kit for amplifying a SARS-CoV nucleotide sequence, which kit comprises:
a) a primer of claim 42; and
b) a nucleic acid polymerase that can amplify a SARS-CoV nucleotide sequence using said primer of claim 42.

51. The kit of claim 50, wherein the nucleic acid polymerase is a reverse transcriptase.

52. An oligonucleotide probe for hybridizing to a SARS-CoV nucleotide sequence, which oligonucleotide probe comprises a nucleotide sequence that:
a) hybridizes, under high stringency, with a target SARS-CoV nucleotide sequence, or a complementary strand thereof, that is set forth in Table 2; or
b) has at least 90% identity to a target SARS-CoV nucleotide sequence comprising a nucleotide sequence, or a complementary strand thereof, that is set forth in Table 2.

53. The probe of claim 52, which comprises DNA, RNA, PNA or a derivative thereof.

54. The probe of claim 52, which comprises a nucleotide sequence, or a complementary strand thereof, that is set forth in Table 2.

55. The probe of claim 52, which is labeled.

56. The probe of claim 55, wherein the label is selected from the group consisting of a chemical, an enzymatic, an immunogenic, a radioactive, a fluorescent, a luminescent and a FRET label.

57. A kit for hybridization analysis of a SARS-CoV nucleotide sequence, which kit comprises:
a) a probe of claim 52; and
b) a means for assessing a hybrid formed between a SARS-CoV nucleotide sequence and said probe.
